(19) United States
(12) Patent Application Publication
(10) Pub. No.: US 2026/0118348 A1
(43) Pub. Date: Apr. 30, 2026

(54) MEASURING RISK OF ADVERSE CLINICAL EVENTS IN CHRONIC LIVER DISEASE

(71) Applicant: HepQuant, LLC, Denver, CO (US)

(72) Inventors: John M. KITTELSON, Missoula, MT (US); Michael P. MCRAE, Houston, TX (US); Gregory Thomas EVERSON, Englewood, CO (US)

(21) Appl. No.: 19/373,222

(22) Filed: Oct. 29, 2025

Related U.S. Application Data
(60) Provisional application No. 63/714,646, filed on Oct. 31, 2024.
(60) Provisional application No. 63/851,099, filed on Jul. 25, 2025.

(51) Int. Cl. G01N 33/53; G01N 33/58; G16H 50/30

(52) U.S. Cl. CPC G01N 33/5308; G01N 33/58; G16H 50/30; G01N 2458/15; G01N 2800/085; G01N 2800/50

(57) ABSTRACT

A method is provided for prediction of risk for adverse clinical outcome in patients with compensated advanced chronic liver disease (cACLD) based on an oral distinguishable cholate challenge test (DuO oral distinguishable cholate liver function test). A predictive model of estimated risk for clinical outcome for communicating cholate challenge test results to patients is also provided.

### CROSS-REFERENCES TO RELATED APPLICATIONS

This application claims the benefit of priority to U.S. Provisional Application No. 63/714,646, filed Oct. 31, 2024 and U.S. Provisional Application No. 63/851,099, filed Jul. 25, 2025, each of which are incorporated by reference herein in their entireties.

### BACKGROUND

### Field of the Invention

Individualization of risk for clinical outcome is a goal of clinical management in patients with chronic liver disease. Early detection of those at risk for liver decompensation to target clinical intervention is crucial for reducing mortality, decreasing healthcare costs, and improving quality of life for patients with compensated advanced chronic liver disease (cACLD).

### Description of the Related Art

Staging of liver fibrosis by liver biopsy and measurement of portal pressure by hepatic venous pressure gradient correlate with risk for clinical outcome. But, both tests are invasive, carry risk for complications, and are not embraced by patients.

Current noninvasive tests for assessment of risk for clinical outcome include standard laboratory tests and elastography, most commonly vibration-controlled-transient-elastography (VCTE). The liver enzymes, AST, ALT, alkaline phosphatase, are markers of liver injury but poorly correlate with risk for clinical outcome. Bilirubin, INR, and albumin reflect liver function but typically become abnormal only at late stages of disease and are often normal in patients with compensated disease. VCTE, although widely adopted, may be less informative in overweight, obese, diabetic patients, especially those with metabolic dysfunction-associated steatotic liver disease (MASLD).

An oral distinguishable cholate challenge test (DuO liver function test) quantifies a disease severity index (DSI), portal-systemic shunting (SHUNT %), and Hepatic Reserve [12-14] and predicts risk for varices [15]. The equivalent SHUNT test correlated with cirrhosis and varices [12, 16], showed improvements after sustained virological response (SVR) [17], and predicted risk for clinical outcomes and hospitalization [18, 19].

The cholate SHUNT liver function test measures portal and systemic clearances simultaneously, comprising: an intravenous (IV) dose of carbon-13-labeled cholate (13C-CA), a simultaneous oral dose of a deuterium-labeled cholate (d4-CA), and 5 peripheral venous blood draws over 90 minutes.

U.S. Pat. No. 8,613,904, Everson et al., discloses cholate SHUNT test methods for evaluating liver function in a patient comprising obtaining patient serum samples following administration of two distinguishable stable isotope labeled cholate compounds, laborious sample processing and analysis of patient serum samples utilizing GC-MS.

U.S. Pat. No. 8,778,299, Everson, discloses cholate SHUNT test methods for evaluating liver function comprising obtaining patient serum samples following administration of two distinguishable stable isotope labeled cholate compounds, processing and analysis of patient serum samples utilizing HPLC-MS. Methods for determining portal hepatic filtration rate (portal HFR, FLOW) from oral administration of a distinguishable agent are also provided.

U.S. Pat. No. 9,091,701, Everson, discloses methods for determining liver function and obtaining a Disease Severity Index (DSI) value in a patient comprising obtaining patient serum samples following administration of two distinguishable stable isotope labeled cholate compounds, processing and analysis of patient serum samples utilizing HPLC-MS.

U.S. Pat. No. 8,961,925, Everson, discloses methods for performing the STAT test comprising oral administration of a distinguishable agent and measuring the distinguishable compound in a single blood or serum sample following using HLPC-MS. Methods of estimating portal hepatic filtration rate (portal HFR) from STAT values are also provided.

US Pat. Appl. Pub. No. US 2021/0318274 A1, Everson et al., discloses improved methods for blood or serum sample preparation, analyte detection and quantification which may be applied to one or more of the SHUNT, FLOW, STAT, and DSI cholate liver function tests.

US Pat. Appl. Pub. No. US 2024/0175881 A1, McRae et al., discloses simplified methods for measuring liver function including DuO oral distinguishable cholate liver function test, for example, using one oral distinguishable cholate dose, and 2 blood draws to obtain orally administered distinguishable cholate concentration at two timepoints which can be used to calculate a DSI value.

One of the challenges in interpreting the cholate challenge test results is that the relationship of DSI value to risk for clinical outcome is nonlinear. The non-linearity of risk predictions means that the risk of adverse events accelerates most rapidly with increasing DSI for patients who have a higher baseline DSI.

### SUMMARY

A method is provided for prediction of risk for clinical outcome in patients with compensated advanced chronic liver disease (cACLD) based on an oral cholate challenge test (DuO liver function test).

A predictive model of estimated risk for clinical outcome (RISK ACE) for communicating cholate challenge test results to patients is provided.

A method is provided for determining risk for an adverse clinical outcome in a patient having a chronic liver disease, the method comprising obtaining a baseline test value from a distinguishable cholate challenge test in the patient; entering the baseline patient test value to a data base comprising comparative distinguishable cholate challenge test values computed from measurements of labeled cholate concentrations in blood samples obtained over time from a known population of chronic liver disease subjects that had been followed prospectively for clinical events; and computing the individual risk for clinical outcome in the patient using the baseline test value as a predictor, optionally wherein the risk for clinical outcome is displayed over a period of time of at least 2 years, at least 3 years, or at least 4 years.

A method is provided for determining risk for an adverse clinical outcome in a patient having a chronic liver disease, the method comprising obtaining a baseline liver disease severity index (DSI) test value from an oral distinguishable cholate challenge test in the patient; entering the baseline patient DSI test value to a data base comprising comparative DSI values computed from measurements of labeled cholate concentrations in blood samples obtained over time from a known population of chronic liver disease subjects that had been followed prospectively for clinical events; and computing the individual risk for clinical outcome in the patient using the baseline DSI value as a predictor, optionally wherein the risk for clinical outcome is displayed over a period of time of at least 2 years, at least 3 years, or at least 4 years.

In some cases, the data base comprising comparative test values was prepared from a Cox proportional hazards regression model, a Cox proportional hazards regression models with time-varying covariates, parametric survival models, or Kaplan Meier, to predict risk for adverse clinical events (RISK ACE) using the comparative distinguishable cholate challenge test values from the distinguishable cholate challenge test.

In some cases, the data base comprising comparative test values was prepared from a Cox proportional hazards regression model to predict risk for adverse clinical events (RISK ACE) using the comparative distinguishable cholate challenge test values from the distinguishable cholate challenge test.

In some cases, the distinguishable cholate challenge test is selected from the group consisting of a liver disease severity index (DSI) test, cholate SHUNT test, an oral only distinguishable cholate challenge test (DuO), Hepatic Reserve test, portal hepatic filtration rate (HFR_{P}), and systemic hepatic filtration rate (HFRs). In some cases, the distinguishable cholate challenge test comprises a DSI test. In some cases, the distinguishable cholate challenge test comprises a cholate SHUNT test. In some cases, the distinguishable cholate challenge test comprises an oral only distinguishable cholate challenge test (DuO).

In some cases, the data base comprises comparative DSI values prepared from a Cox proportional hazards regression model to predict risk for adverse clinical events (RISK ACE) using the disease severity index (DSI) values from the oral distinguishable cholate challenge test.

In some cases, the DSI test values are obtained from an oral only distinguishable cholate challenge test (DuO). In some cases, the DSI test values are obtained from cholate SHUNT test values. In some cases, the DSI values are obtained from a portal hepatic filtration rate (HFR_{P}), and a systemic hepatic filtration rate (HFRs).

In some cases, the patient is suffering from a compensated advanced chronic liver disease (cACLD).

In some cases, the clinical outcome is selected from the group consisting of liver-related death, hepatocellular carcinoma (HCC), Child-Pugh progression, variceal hemorrhage, ascites, hepatic encephalopathy, and other manifestations of hepatic decompensation. In some cases, other manifestations of hepatic decompensation are selected from the group consisting of jaundice, coagulopathy, nutritional deficiencies, muscle wasting, sepsis, and spontaneous bacterial peritonitis. In some cases, the clinical outcome is selected from the group consisting of liver-related death, hepatocellular carcinoma (HCC), Child-Pugh progression, variceal hemorrhage, ascites, and hepatic encephalopathy.

In some cases, the comparative test values were collected in at least two different time points from the population of chronic liver disease subjects. In some cases, the comparative test values were collected in at least two different time points from the population of known chronic liver disease subjects.

In some cases, the method further comprises obtaining a follow-up distinguishable cholate challenge test value in the patient after a first period of time and entering the follow-up test value to the data base comprising the comparative test values; entering date the baseline test value was obtained and date the follow-up test value was obtained; and computing the individual risk for clinical outcome in the patient using the baseline test value, and subsequent change from baseline in the follow-up test value as predictors.

In some cases, the method further comprises obtaining a follow-up distinguishable cholate challenge test value in the patient and entering the follow-up test value to the data base comprising the comparative test values; entering date the baseline test value was obtained and date the follow-up test value was obtained; and computing the individual risk for clinical outcome in the patient using the baseline test value, and the follow-up test value as predictors.

In some cases, the method further comprises obtaining a follow-up distinguishable cholate challenge test value in the patient and entering the follow-up test value to the data base comprising the comparative test values; entering the dates the baseline and follow-up test values were obtained in the patient; and computing the individual risk for clinical outcome in the patient using the baseline test value and change between the baseline and follow-up test values divided by the time in months between the baseline test and follow-up test as predictors.

In some cases, the distinguishable cholate compound is an isotope labeled distinguishable cholate compound. In some cases, the distinguishable cholate compound is a stable isotope labeled distinguishable cholate compound. In some cases, the stable isotope labeled cholate compound is selected from the group consisting of d4-cholate, d5-cholate, d2 cholate, and ¹³C-cholate, optionally wherein the d4-cholate is 2,2,4,4-d4 cholate, optionally wherein the d5-cholate is 2,2,3,4,4-cholate, and further optionally wherein the 13C cholate is 24-¹³C-cholate.

In some cases, the chronic liver disease is selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

In some cases, the patient or subject is a human patient or subject.

In some cases, the method comprises obtaining blood or serum sample concentration data of an orally administered distinguishable cholate compound collected from the patient at two time points after oral administration; measuring the area under the curve of the blood or serum concentrations of the orally administered distinguishable cholate compound (AUCoral) in the patient comprising simulating a full oral clearance curve using a compartmental model of oral cholate clearance, the compartmental model comprising body mass index (BMI), body weight (BW), and optionally hematocrit (Hct) input values in the patient, and calculating the area comprising trapezoidal numerical integration to obtain the AUCoral. In some cases, the method further comprises calculating one or more distinguishable cholate challenge test results in the patient using the AUCoral, wherein the test results are associated with liver function in the patient.

In some cases, the obtaining concentration data of the administered distinguishable cholate compound at the two time points comprises receiving first and second blood or serum samples that had been collected from the patient at first and second time points following a single oral dose of a first distinguishable cholate compound; and analyzing the samples to obtain the oral concentration data at the first and second time points. In some cases, the blood or serum samples had been collected within about 180 minutes, 120 minutes, 90 minutes, or within about 75 minutes, after the oral administration.

In some cases, the first and second blood or serum samples had been collected from the subject between at least about 5 min to about 75 min, 10 min to 70 min, 20 min to 60 min, 25 min to 55 min, 30 min to 50 min, 35 to 45 min, or about 40 min apart. In some cases, the first and second blood or serum samples had been collected from the patient at about 20 min and about 60 min following the oral administration, respectively.

In some cases, the method further comprises estimating an area under the curve of blood or serum concentrations of an intravenously administered distinguishable cholate compound (AUCiv); and calculating a DSI value or SHUNT test value in the patent using the AUCoral and estimated AUCiv values.

In some cases, the estimating the AUCiv comprises a linear regression model, optionally wherein the linear regression model comprises equation 11A:

$${AUC}_{IV} = {β}_{0} + {β}_{BW} \cdot BW + {β}_{PO , 20} \cdot {C}_{PO , 20} + {β}_{PO , 60} \cdot {C}_{PO , 60} + {β}_{HFR , P} \cdot {HFR}_{P} , \qquad Eqn . 11 A ,$$

wherein
β₀ is an intercept coefficient, optionally wherein the intercept coefficient is 161.972; B_{BW} is a body weight coefficient, optionally wherein the body weight coefficient is 0.6459;
β_{PO,20} is an orally administered distinguishable cholate concentration coefficient at a first time point, optionally wherein the β_{PO,20} is 16.9249;
C_{PO,20} is the orally administered distinguishable cholate concentration at the first time point;
β_{PO,60} is an orally administered distinguishable cholate concentration coefficient at a second time point, optionally wherein the β_{PO,60} is 89.2405;
C_{PO,60} is an orally administered distinguishable cholate concentration at the second time point; and
β_{HFR, P} is a portal HFR coefficient, optionally wherein the portal HFR coefficient is −0.4755.

In some cases, the estimating the AUCiv comprises exponential fitting the intravenous concentration data to a systemic cholate clearance curve comprising fast, moderate, and slow phases of clearance over at least about 180 min after iv administration of an intravenous dose.

In some cases, the fitting to systemic cholate clearance curve fast phase (Y₀) is calculated according to equation 20:

$${Y}_{0} = {C}_{0} \cdot {e}^{- {k}_{fast} \cdot t} , \qquad Eqn . 20$$

wherein t=time (0 to 20 min); C₀ is the initial concentration of intravenously administered distinguishable cholate compound, C₂₀ is the measured 20-minute concentration of intravenously administered distinguishable cholate compound; and k_{fast} is the rate of elimination in the fast phase estimated by equation 18:

$${k}_{fast} = - \frac{ln ( {C}_{20} ) - ln ( {C}_{0} )}{{T}_{20}} . \qquad Eqn . 18$$

T₂₀ is the actual time recorded for the 20-minute sample. C₀ is estimated according to equation 17;

$${C}_{0} = \frac{{D}_{IV}}{{V}_{d} \cdot BW} , \qquad Eqn . 17$$

wherein D_{IV} is the intravenous dose of third distinguishable cholate; BW is subject body weight (kg); and Vd is the volume of distribution (V_{d}) in L per kg body weight, calculated according to equation 16A:

$$TPV = {V}_{d} = \frac{0.07}{\sqrt{BMI / 22}} \cdot ( 1 - Hct ) , \qquad Eqn . 16 A$$

wherein TPV is total plasma volume, BMI is body mass index, and Hct is hematocrit in the subject.

In some cases, the fitting to systemic cholate clearance curve moderate phase (Y₁) is calculated according to equation 21:

$${Y}_{1} = \frac{{C}_{20}}{{e}^{- {k}_{mod} \cdot 20}} \cdot {e}^{- {k}_{mod} \cdot t} , \qquad Eqn . 21$$

wherein t=time (20-60 min); k_{mod} is the rate of elimination in the moderate phase estimated by equation 19:

$${k}_{mod} = - \frac{ln ( {C}_{60} ) - ln ( {C}_{20} )}{{T}_{60} - {T}_{20}} , \qquad Eqn . 19$$

wherein T₂₀ is the actual time recorded for the 20-minute sample; T₆₀ is the actual time recorded for the 60-minute sample; C₂₀ is the concentration at 20 minutes; and C₆₀ is the concentration at 60 minutes.

In some cases, the fitting to systemic cholate clearance curve slow phase (Y₂) is calculated according to equation 22:

$${Y}_{2} = \frac{{C}_{60}}{{e}^{- {k}_{slow} \cdot {T}_{60}}} \cdot {e}^{- {k}_{slow} \cdot t} , \qquad Eqn . 22$$

wherein t=time (60-180 min); C₆₀ is the 60-minute concentration of intravenously administered distinguishable cholate; and k_{slow} is the rate of elimination in the slow phase estimated by a mean value from a multiplicity of CLD patients, optionally wherein k_{slow} is 0.018 min⁻¹.

In some cases, the areas under each of the three exponential curve fits are calculated by trapezoidal numerical integration and summed to estimate the AUC_{IV}.

A computer program product is provided, comprising a non-transitory and tangible computer-readable storage medium whose contents include a program with instructions being executed on a processor so as to perform a method for determining risk for an adverse clinical outcome in a patient having a chronic liver disease, the method comprising obtaining a baseline test value from a distinguishable cholate challenge test in the patient; entering the baseline patient test value to a data base comprising comparative distinguishable cholate challenge test values computed from measurements of labeled cholate concentrations in blood samples obtained over time from a known population of chronic liver disease subjects that had been followed prospectively for clinical events; and computing the individual risk for clinical outcome in the patient using the baseline test value as a predictor, optionally wherein the risk for clinical outcome is displayed over a period of time of at least 2 years, at least 3 years, or at least 4 years.

A method is provided for analyzing a patient response to a treatment of a chronic liver disease, the method comprising determining a baseline cholate Disease Severity Index (DSI) value in the patient having a chronic liver disease prior to the treatment; administering the treatment to the patient for a period of time; and determining a follow-up cholate Disease Severity Index (DSI) value in the patient after the period of treatment time, wherein a treatment responder exhibits >2-point decrease in the follow-up DSI value, a stable subject exhibits a ΔDSI within ±2-points, and a non-responder exhibits >2-point increase compared to the baseline DSI value. In some cases, the period of time is from 2 weeks to 104 weeks, 4 weeks to 78 weeks, 8 weeks to 52 weeks, or 28 weeks to 48 weeks.

In some cases, the determining the baseline cholate DSI value in the patient comprises obtaining blood or serum sample concentration data of an orally administered distinguishable cholate compound collected from the patient at two time points after oral administration; measuring the area under the curve of the blood or serum concentrations of the orally administered distinguishable cholate compound (AUCoral) in the patient comprising simulating a full oral clearance curve using a compartmental model of oral cholate clearance, the compartmental model comprising body mass index (BMI), body weight (BW), and optionally hematocrit (Hct) input values in the patient, and calculating the area comprising trapezoidal numerical integration to obtain the AUCoral; estimating an area under the curve of blood or serum concentrations of an intravenously administered distinguishable cholate compound (AUCiv); and calculating the DSI value in the patent using the AUCoral and estimated AUCiv values.

In some cases, the obtaining concentration data of the orally administered distinguishable cholate compound at the two time points comprises receiving first and second blood or serum samples that had been collected from the patient at first and second time points following a single oral dose of a first distinguishable cholate compound; and analyzing the samples to obtain the oral concentration data at the first and second time points, optionally wherein the blood or serum samples had been collected within about 180 minutes, 120 minutes, 90 minutes, within about 75 minutes, or within about 60 minutes after the oral administration.

In some cases, the first and second blood or serum samples had been collected from the subject between at least about 5 min to about 90 min, 10 min to 75 min, 20 min to 60 min, 25 min to 55 min, 30 min to 50 min, 35 to 45 min, or about 40 min apart. In some cases, the first and second blood or serum samples had been collected from the subject at about 20 min and about 60 min following the oral administration, respectively.

### BRIEF DESCRIPTION OF THE DRAWINGS

FIG. 1 shows a graph of predicted probability of adverse clinical event (%) vs. time from baseline test, using baseline DSI score in patients as the only predictor. Predicted risk for an adverse clinical event (RISK ACE) by quartiles of DSI within the indicated time based on the disease severity index (DSI) at baseline (Model A) is compared to the Kaplan-Meier estimated risk (solid line). Model A is intended to be used in interpreting the results of a single cholate challenge test (DuO oral distinguishable cholate challenge test) (e.g., a patient's first test).

FIG. 2A-D shows four graphs of evaluation of predicted risk for adverse clinical events (RISK ACE) with various changes in disease severity index (DSI) after approximately two years follow-up. The baseline model (Model A, solid black line) was evaluated with baseline DSI values of DSI 18.3 (FIG. 2A), DSI 23 (FIG. 2B), DSI 30 (FIG. 2C), and DSI 40 (FIG. 2D), with predicted probabilities expressed in terms of time from the baseline test. The rate of change model (Model D, dashed lines) was evaluated using the corresponding baseline DSI values and with various changes from baseline at follow-up, with predicted probabilities expressed in terms of time from the follow-up test. These results suggest that progression of liver disease, as measured by an increase in the rate of change of DSI, has a greater effect on clinical outcome risk in those with higher baseline DSI.

FIG. 3 shows a representative screenshot of a RISK ACE web app (Baseline, Model A). Patients and/or providers input DSI results, and the RISK ACE results are updated dynamically in the plots and tables. The web application (app) facilitates the use and explanation of the RISK ACE evaluation of the results of liver function testing. The web app was developed using the R package, Shiny, and can be accessed by smart phone or computer. The Baseline Model evaluates the RISK ACE scores (i.e., the predicted risk at baseline for the occurrence of an adverse clinical event from Cox proportional hazards regression) using DSI at baseline and is intended to be used in interpreting the results of a single HepQuant DuO test (e.g., a patient's first test). The only input for the Baseline Model is DSI (left panel). The markers plotting the RISK ACE score over time may change color depending on the DSI value (for example, DSI<18.3, green (open circles); DSI 18.3 to 23, yellow/orange (grey circles); DSI>23, red)(solid circles).

FIG. 4 shows a representative screenshot of RISK ACE web app (Follow-up, Model D). Patients and providers input DSI results, and the RISK ACE results are updated dynamically in the plots and tables. The web application (app) facilitates the use and explanation of the RISK ACE evaluation of the results of liver function testing. The web app was developed using the R package, Shiny, and can be accessed by smart phone or computer. The Follow-Up Model evaluates the RISK ACE scores (i.e., the predicted risk at follow-up for the occurrence of an adverse clinical event from Cox proportional hazards regression) using DSI at baseline and the rate of change from baseline (i.e., the change in DSI divided by the time in months between baseline and follow-up tests) and is intended for monitoring applications to track liver function over time. The inputs for the Follow-Up Model are (1) baseline DSI, (2) date of baseline test, (3) follow-up DSI, and (4) date of follow-up test. A calendar widget facilitates the selection of dates. The markers plotting the RISK ACE score over time may change color depending on the follow-up DSI value (for example, DSI<18.3, green (open circles); DSI 18.3 to 23, yellow/orange (grey circles); DSI>23, red (solid circles)).

FIG. 5 shows Table 9 with Disease Severity Index (DSI) values from the DuO oral distinguishable cholate challenge test at each stage of fibrosis from HALT-C patients who developed clinical outcomes and subjects without clinical outcomes.

FIG. 6 shows Table 10 with SHUNT % from the DuO oral distinguishable cholate challenge test at each stage of fibrosis from HALT-C patients who developed clinical outcomes and subjects without clinical outcomes.

FIG. 7 shows Table 11 with Hepatic Reserve (%) from the DuO oral distinguishable cholate challenge test at each stage of fibrosis from HALT-C patients who developed clinical outcomes and subjects without clinical outcomes.

FIG. 8 shows probability of adverse clinical events by RISK ACE before and after 48 weeks of resmetirom for all subjects (n=23). The solid line represents the baseline DSI and probability (%) of clinical outcome prior to treatment with resmetirom. Dashed line represents the effect after 48 weeks treatment with resmetirom on probability (%) of clinical outcome risk based on week 48 DSI measurement. RISK ACE decreased after week 48 of treatment in 19 of 23 subjects, with a significant reduction in absolute risk of −4.0%, p=0.041.

FIG. 9 shows the probability of adverse clinical events by RISK ACE for all subjects with follow-up at 48 weeks treatment with resmetirom and stratified by baseline DSI (n=23), with DSI>23 indicating high risk, DSI 18.3-23 indicating moderate risk, and DSI<18.3 indicating low risk.

FIG. 10 shows two waterfall plots of change in cholate disease severity index (DSI) from baseline in subjects taking 75 mg, 150 mg, or 225 mg rencofilstat daily measured by DuO cholate challenge test at Day 60 (Panel A) and Day 120 (Panel B). The horizontal lines represent limits of ±2 DSI units for responder or worsening thresholds.

### DETAILED DESCRIPTION OF THE INVENTION

### Definitions

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of +/−10% of the specified amount. In some cases, the term “about” refers to, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

As used herein “clearance” may mean the removing of a substance from one place to another.

As used herein, the term “simultaneously” when referring to 2 or more events refers to occurring within 10 minutes or less, 5 minutes, or within about 3 minutes of each other.

As used herein the terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles. In a particular aspect, the patient or subject is human.

The term “adverse clinical outcome” or “clinical outcome” as used herein refers to a medical event that occurs as a result of a chronic liver disease. In some cases, the clinical outcome is selected from the group consisting of liver-related death, hepatocellular carcinoma (HCC), Child-Pugh progression, MELD progression, development of varices in patient without prior varices, development of large varices with prior no or small varices, variceal hemorrhage, ascites, hepatic encephalopathy, and other manifestations of hepatic decompensation. In some cases, other manifestations of hepatic decompensation are selected from the group consisting of jaundice, coagulopathy, nutritional deficiencies, muscle wasting, sepsis, and spontaneous bacterial peritonitis. In some cases, the clinical outcome is selected from the group consisting of liver-related death, hepatocellular carcinoma (HCC), Child-Pugh progression, variceal hemorrhage, ascites, and hepatic encephalopathy.

The term “cholate challenge test” refers to a liver function test comprising administering one or more distinguishable cholate compounds to a subject or patient having or suspected of having or contracting a chronic liver disease, quantifying the one or more distinguishable cholates in the blood or serum of the subject or patient at one or more, or two or more time points after administration, and processing the distinguishable cholate data to obtain liver function test data. In some cases, the cholate challenge test comprises obtaining blood or serum of a subject or patient at one or more, or two or more time points after administration of one or more distinguishable cholate compounds, quantifying the distinguishable cholate in the blood or serum of the subject or patient at one or more, or two or more time points after administration of one or more distinguishable cholate compounds to the patient or subject, and processing the distinguishable cholate data to obtain liver function test data. In some cases, the cholate challenge test comprises obtaining concentration data of one or more distinguishable cholate compounds in the blood or serum obtained from a subject or patient at one or more, or two or more time points after administration of the one or more distinguishable cholates, and processing the distinguishable cholate concentration data to obtain liver function test data. In some cases, the cholate challenge test can be selected from the group consisting of a cholate SHUNT test, DuO oral distinguishable cholate challenge test, liver Disease Severity Index (DSI) test, Hepatic Reserve (%), cholate STAT test, portal hepatic filtration rate (portal HFR), systemic hepatic filtration rate (systemic HFR), and RCA20. In some cases, cholate SHUNT test results can be used to calculate DSI, SHUNT % (%)(portal-systemic shunting), Hepatic Reserve (%), portal HFR (mL/min/kg), or systemic HFR (mL/min/kg), from cholate SHUNT V1.0, SHUNT V1.1, or SHUNT V2.0. In some cases, the cholate SHUNT test can be selected from the group consisting of a cholate SHUNT V1.0 liver function test, a cholate SHUNT V1.1 liver function test, and a cholate SHUNT V2.0 liver function test.

The acronym “HALT-C” refers to the Hepatitis C Antiviral Long-term Treatment against Cirrhosis trial. The HALT-C trial was a large, prospective, randomized, controlled trial of long-term low dose peg interferon therapy in patients with advanced hepatitis C who had not had a sustained virologic response to a previous course of interferon-based therapy. The HALT-C Trial examined whether long-term use of antiviral therapy (maintenance treatment) would slow the progression of liver disease. In noncirrhotic patients who exhibited significant fibrosis, effective maintenance therapy was expected to slow or stop histological progression to cirrhosis as assessed by serial liver biopsies. However, tracking disease progression with biopsy carries risk of complication, possibly death. In addition, sampling error and variation of pathologic interpretation of liver biopsy limits the accuracy of histologic assessment and endpoints. The histologic endpoint is less reliable because advanced fibrosis already exists and changes in fibrosis related to treatment or disease progression cannot be detected. Thus, standard endpoints for effective response to maintenance therapy in cirrhotic patients are prevention of clinical decompensation (ascites, variceal hemorrhage, and encephalopathy) and stabilization of liver function as measured clinically by Childs-Turcotte-Pugh (CTP) score. However, clinical endpoints and CTP score were known to be insensitive parameters of disease progression. Dual isotope techniques employing distinguishable cholates were used in development of the SHUNT test and used in conjunction with the HALT-C trial.

The term “SHUNT test” or “cholate SHUNT test” refers to a previously disclosed QLFT (quantitative liver function test) used as a comprehensive assessment of hepatic blood flow and liver function. The cholate SHUNT test is used to determine clearance of orally and intravenously administered distinguishable cholic acids in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of at least about 90 minutes after oral and intravenous administration of differentiable cholates. The SHUNT test is disclosed in Everson et al., U.S. Pat. No. 8,613,904, which is incorporated herein by reference. These studies demonstrated reduced clearance of cholate in patients who had either hepatocellular damage or portosystemic shunting. The “SHUNT test value” refers to a number (in %).

The term “SHUNT %” represents a quantitative measurement of portal-systemic shunting. SHUNT % is the portal-systemic shunt fraction, which is defined as the ratio of the systemic and portal clearances. In some cases, SHUNT % is a measurement of the percentage of spillover of the orally administered d4-cholate. The first-pass hepatic elimination of cholate in percent of orally administered cholate is defined as (100%—SHUNT). SHUNT test methods are disclosed in U.S. Pat. Nos. 8,613,904, 9,639,665, 8,778,299, 9,417,230, and 10,215,746, each of which is incorporated herein by reference in its entirety. Analysis of samples for stable isotopically labeled cholates is performed by, e.g., GC-MS, following sample derivitization, or LC-MS, without sample derivitization, or LC-MS/MS, or MS/MS as disclosed herein. The ratio of the AUCs of orally to intravenously administered cholic acid, corrected for administered doses, defines cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally administered cholic acid and AUCᵢᵥ is the area under the curve of the intravenously administered cholic acid.

The SHUNT test allows measurement of first-pass hepatic elimination of bile acids from the portal circulation. Flow-dependent, first pass elimination of bile acids by the liver ranges from about 60% for unconjugated dihydroxy, bile acids to about 95% for glycine-conjugated cholate. Free cholate, used herein has a reported first-pass elimination of approximately 80% which agrees closely with previously observed first pass elimination in healthy controls of about 83%. After uptake by the liver, cholic acid is efficiently conjugated to either glycine or taurine and secreted into bile. Physicochemically cholic acid may be easily separated from other bile acids and bile acid or cholic acid conjugates, using chromatographic methods.

The “DuO oral distinguishable cholate challenge test is a liver function test”, also known as Dual Sample Oral Cholate Challenge Test (also known as “DuO”, “DUO”, “DuO test”, “DuO cholate test”, “HepQuant DUO”, “HepQuant DuO”) is a compartmental model of portal cholate clearance that uses assumptions of liver flow and physiology to predict oral clearance curves comprising measuring only two (e.g., 20 min. and 60 min) orally administered distinguishable cholate (e.g., d4-CA) concentration timepoints (i.e., DuO liver function test v1.0). The DuO v1.0 test comprises administration of 1 oral dose of a distinguishable cholate (e.g., d4-CA at 0 min.), and collection of 2 blood draws at first and second time points (e.g., 20 and 60 min.). The DuO v2.0 test comprises administration of 2 oral doses of first and second distinguishable cholates, respectively, at first and second time points (e.g., d4-CA at 0 min., 13C-CA at 40 min.), and collection of 1 blood draw at a single time point (e.g., 60 min.). The DuO test quantifies portal HFR and estimates systemic HFR, DSI, SHUNT %, and Hepatic Reserve (HR) using only oral dose(s). The DuO dual oral cholate clearance tests do not require measurement of an intravenously administered distinguishable cholate.

The liver Disease Severity Index (DSI) can be calculated according to equation 14:

$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} , \qquad Eqn . 14$$

wherein HFR_{P,max} is the upper limit of portal clearance from a multiplicity of healthy controls; HFR_{S,max} is the upper limit of clearance from a multiplicity of healthy controls; and A is a factor to scale DSI from 0 to 50.

The DuO test (also known as dual sample oral cholate challenge test) is an oral-only cholate liver function test involving administration of one oral dose (d4-CA at 0 minutes) and collection of two blood samples (e.g., at 20 and 60 minutes) to quantify portal HFR and estimate systemic HFR. These analyses involve the same compartmental model of portal cholate clearance as SHUNT V2.0. The systemic clearance is calculated by first estimating the derived IV concentrations at 20 and 60 minutes using linear regression models with body weight, BMI, the actual time of the 20-minute blood sample, and d4-CA concentrations at 20 and 60 minutes (see US 2024/0175881 A1, McRae et al., for a detailed description of the linear models and the regression coefficients). The derived IV concentrations are then used in the same noncompartmental analysis as SHUNT V2.0.

The cholate SHUNT V1.0 liver function test and Minimal Model (MM) analysis method describes IV clearance by noncompartmental exponential fits and oral clearance by a cubic spline fit to calculate the areas under the IV and oral curves (AUC_{IV}, AUC_{Oral}), respectively. Everson et al. Portal-systemic shunting inpatients with fibrosis or cirrhosis due to chronic hepatitis C: the minimal model for measuring cholate clearances and shunt. Aliment Pharmacol Ther. 2007; 26:401-10. Peripheral venous blood is obtained at 0, 5, 20, 45, 60, and 90 minutes within specified time windows, and concentrations of oral and intravenous administered distinguishable cholates are measured by liquid chromatography/mass spectrometry (LC/MS). After IV injection, residual 13C-CA in the sampling catheter and sampling blood beyond the time window, especially for the 5-minute sample (±1 minute), were sources of error in a minority of tests but may have compromised results in those tests.

The cholate SHUNT V1.1 liver function test is a cholate liver function test that employs an MM_{Vd} (i.e., Minimal Model with initial concentration derived from estimated volume of distribution) analysis method to eliminate the 5-minute sample from the calculations. The initial 13C-CA concentration at 0 minutes is calculated from administered dose and estimation of blood volume from body mass index (BMI). Lemmens et al., Estimating blood volume in obese and morbidly obese patients. Obes Surg. 2006; 16:773-6. The 5-minute 13C-CA concentration is then estimated by log-linear regression between the 0- and 20-minute concentrations, and the 5-minute oral concentration is approximated as 15% of the 20-minute d4-CA concentration. The AUC_{IV} and AUC_{Oral} are then estimated by the same Minimal Model equations as in SHUNT V1.0.

The cholate SHUNT V2.0 is a cholate liver function test that implements (1) a compartmental model of portal cholate clearance that uses assumptions of liver flow and physiology to predict oral clearance curves and (2) noncompartmental exponential fits to systemic cholate clearance. The compartmental model for measuring portal clearance describes the flow between systemic (S), portal (P), and liver (L) compartments represented by a system of first-order ordinary differential equations (Equations 1B, 2B, and 3), where q is the flow rate between compartments, V is the volume of the compartment, C is the concentration of d4-CA in the compartment, Cl_{H} is the hepatic clearance, and D_{PO,rate} is the rate of orally administered d4-CA entering the portal compartment.

$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) \qquad Eqn . 1 B$$

$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) \qquad Eqn . 2 B$$

$$\frac{{dC}_{L}}{dt} = \frac{1}{{V}_{L}} ( - {Cl}_{H} \cdot {C}_{L} - {q}_{LS} \cdot {C}_{L} + {q}_{PL} \cdot {C}_{P} + {q}_{SL} \cdot {C}_{S} ) \qquad Eqn . 3$$

The system of ordinary differential equations is solved numerically, and the concentration of d4-CA in the systemic compartment is integrated over 180 minutes to calculate the AUC_{Oral}.

The noncompartmental analysis for measuring systemic clearance involves the exponential fits of systemic cholate clearance using the 20- and 60-minute 13C-CA concentrations. The exponential fits split the curve into three clearance phases: fast (Y₀), moderate (Y₁), and slow (Y₂). Equations 4C, 5B, and 6B define the systemic concentration of 13C-CA through time.

$${Y}_{0} = {C}_{0} \cdot {e}^{- {k}_{fast} \cdot t} \qquad Eqn . 4 C$$

$${Y}_{1} = \frac{{C}_{20}}{{e}^{- {k}_{mod} \cdot {T}_{20}}} \cdot {e}^{- {k}_{mod} \cdot t} \qquad Eqn . 5 B$$

$${Y}_{2} = \frac{{C}_{60}}{{e}^{- {k}_{slow} \cdot {T}_{60}}} \cdot {e}^{- {k}_{slow} \cdot t} \qquad Eqn . 6 B$$

Here, t is time (0-20, 20-60, and 60-180 minutes for Y₀, Y₁, and Y₂, respectively), C₀ is the initial concentration of 13C-CA, C₂₀ and C₆₀ are the measured 20- and 60-minute concentrations of 13C-CA, and T₂₀ and T₆₀ are the actual collection times of the 20- and 60-minute blood sample. The systemic concentration of 13C-CA is then integrated to calculate the AUC_{IV}. See the Example 2 for a more detailed description of both the compartmental analysis for portal clearance and the noncompartmental analysis for systemic clearance.

The DuO test (also known as DuO V1.0 or dual sample oral cholate challenge test) is an oral-only cholate liver function test involving administration of one oral dose (d4-CA at 0 minutes) and collection of two blood samples (e.g., at 20 and 60 minutes) to quantify portal HFR and estimate systemic HFR. These analyses involve the same compartmental model of portal cholate clearance as SHUNT V2.0. The systemic clearance is calculated by first estimating the derived IV concentrations at 20 and 60 minutes using linear regression models (Table 1A) with body weight, BMI, the actual time of the 20-minute blood sample, and d4-CA concentrations at 20 and 60 minutes (see Example 2 for a detailed description of the linear models and the regression coefficients). The derived IV concentrations are then used in the same noncompartmental analysis as SHUNT V2.0.

The term “Cholate Elimination Rate”, kₑₗᵢₘ min⁻¹ represents the first phase of elimination of the intravenously administered 13C-cholate, calculation from Ln/linear regression of [13C-cholate]versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, V_{d}, (L kg⁻¹) represents the body's volume into which cholate is distributed.

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The acronym “ROC” refers to receiver operating characteristic. The ROC curve is a graphical plot which illustrates performance of a binary classifier system as its discrimination threshold is varied. It is created by plotting the fraction of true positives out of the positives (TPR=true positive rate) vs. the fraction of false positives out of the negatives (FPR=false positive rate), at various threshold settings. Sensitivity is the probability of a positive test result, or of a value above a threshold, among those with disease. Sensitivity is defined as the true positive rate (TPR): TPR=TP/P=TP/(TP+FN). False positive rate (FPR) is FPR=FP/N=FP/(FP+FN). Accuracy (ACC) is defined as ACC=(TP+TN)/(P+N). Specificity is the probability of a negative test result, or a value below a threshold, among those without disease. Specificity (SPC), or true negative rate (TN) is defined as SPC=TN/N=TN/(FP+TN)=1-FPR. Positive prediction value (PPV) is defined as: PPV=TP/(TP+FP). Negative predictive value (NPV) is defined as NPV=TN/(TN+FN).

The c-statistic is the area under the ROC curve, or “AUROC” (area under receiver operating characteristic curve) and ranges from 0.5(no discrimination) to a theoretical maximum of 1(perfect discrimination).

The terms “treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “sustained virologic response” (SVR) is used to describe a desired response in a patient when, e.g., hepatitis C virus is undetectable in the blood six months after finishing treatment. Conventional treatment using interferon and ribavirin doesn't necessarily eliminate, or clear, the hepatitis C virus. A sustained virologic response is associated with a very low incidence of relapse. SVR is used to evaluate new medicines and compare them with proven therapies.

The term “distinguishable cholate” or “distinguishable cholate compound” may be may any cholate compound that is distinguishable analytically from naturally occurring cholate in the blood or serum of a subject. The distinguishable cholate compound may be a labeled cholate compound or an unlabeled cholate compound. The distinguishable cholate compound may be a fluorescent moiety-labeled cholate compound. Various fluorescent probes are commercially available such as, e.g., fluorescein, Alexa Fluor dyes, quantum dots, and the like. The distinguishable cholate be an isotope labeled cholate compound. Distinguishable cholate compounds may be labeled with either stable isotopes (e.g., ¹³C, ²H, ¹⁸O) or radioactive isotopes (e.g., ¹⁴C, ³H). Distinguishable cholate compounds are commercially available and can be purchased (for example CDN Isotopes Inc., Quebec, CA).

The distinguishable cholate compounds may be stable isotope labeled cholate compounds. The distinguishable cholate may be selected from any known safe, non-radioactive stable isotope of cholic acid. In one specific aspect, the distinguishable cholate compound is 2,2,4,4-²H cholic acid, also known as cholic-acid-2,2,4,4-d₄ (D₄-CA). In another specific aspect, the distinguishable cholate compound is 24-¹³C cholic acid, also known as cholic acid-24-¹³C (¹³C-CA). In another specific aspect, the distinguishable compound is 2,2,3,4,4-²H cholic acid, also known as cholic acid-2,2,3,4,4-d₅ (D₅-CA).

In some embodiments, the distinguishable cholate compound may be selected from any of the following labeled compounds: cholic acid, any glycine conjugate of cholic acid, any taurine conjugate of cholic acid; chenodeoxycholic acid, any glycine conjugate of chenodeoxycholic acid, any taurine conjugate of chenodeoxycholic acid; deoxycholic acid, any glycine conjugate of deoxycholic acid, any taurine conjugate of deoxycholic acid; or lithocholic acid, or any glycine conjugate or taurine conjugate thereof. The distinguishable cholate compound may be selected from those described in WO 2021/207683 A1, HepQuant, LLC, Everson and Helmke, which is incorporated herein by reference in its entirety.

Cholates occur naturally and are not known to have any deleterious or adverse effects when given intravenously or orally in the doses used in the inventive or comparative tests herein. The serum cholate concentrations that are achieved by either the intravenous or oral doses are similar to the serum concentrations of bile acids that occur after the ingestion of a fatty meal. Because cholates are naturally occurring with a pool size in humans of 1 to 5 g, the 20 and 40 mg doses of labeled cholates used herein are unlikely to be harmful.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” (HFRp) refers to oral cholate clearance (portal hepatic filtration rate; portal HFR) used as a measure of portal blood flow, or portal circulation, obtained from analysis of concentration of distinguishable cholate compound in at least 5 blood samples drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL min⁻¹ kg⁻¹ may be used to Model independent apparent clearance of orally administered d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety.

The following metrics can be measured by the DuO distinguishable cholate liver function test and have previously demonstrated associations with liver function. DuO can be performed by any method known in the art, for example, US 2024/0175881 A1, McRae et al., which is incorporated herein by reference in its entirety. The systemic clearance is calculated by first estimating the derived IV concentrations at 20 and 60 minutes using linear regression models (Table 1A) with body weight, BMI, the actual time of the 20-minute blood sample, and d4-CA concentrations at 20 and 60 minutes. The derived IV concentrations are then used in the same noncompartmental analysis as SHUNT V2.0.

For the oral-only simplified liver function test, DuO, the derived IV 20- and 60-minute concentrations can be estimated by Equation S16 and Equation S17.

$${C}_{IV ( 20 )} = {β}_{0 , 20} + {β}_{BW , 20} \cdot ( \frac{BW - {Z}_{BW , mean}}{{Z}_{BW , SD}} ) + {β}_{BMI , 20} \cdot ( \frac{BMI - {Z}_{BMI , mean}}{{Z}_{BMI , SD}} ) + {β}_{PO ( 20 ) , 20} \cdot ( \frac{ln [ {C}_{PO ( 20 )} ] - {Z}_{PO ( 20 ) , mean}}{{Z}_{PO ( 20 ) , SD}} ) + {β}_{PO ( 60 ) , 20} \cdot ( \frac{ln [ {C}_{PO ( 60 )} ] - {Z}_{PO ( 60 ) , mean}}{{Z}_{PO ( 20 ) , SD}} ) + {β}_{T 20} \cdot ( \frac{{T}_{20} - {Z}_{T 20 , mean}}{{Z}_{T 20 , SD}} ) \qquad Eqn . S16$$

$${C}_{IV ( 60 )} = {β}_{0 , 60} + {β}_{BW , 60} \cdot ( \frac{BW - {Z}_{BW , mean}}{{Z}_{BW , SD}} ) + {β}_{PO ( 20 ) , 60} \cdot ( \frac{ln [ {C}_{PO ( 20 )} ] - {Z}_{PO ( 20 ) , mean}}{{Z}_{PO ( 20 ) , SD}} ) + {β}_{PO ( 60 ) , 60} \cdot ( \frac{ln [ {C}_{PO ( 60 )} ] - {Z}_{PO ( 60 ) , mean}}{{Z}_{PO ( 60 ) , SD}} ) \qquad Eqn . S17$$

The linear models can be trained using data from the SHUNT-V Study (ClinicalTrials.gov. The SHUNT-V Study for Varices. clinicaltrials.gov/ct2/show/NCT03583996. Accessed Jul. 20, 2022.) (N=275) and healthy controls (N=50) and resulted in the standardization constants and regression coefficients listed in Table 1A. The AUC_{IV} for DuO can then calculated by the same method as SHUNT V2.0, using instead estimated 20- and 60-minute IV concentrations rather than measured values.

TABLE 1A
Linear regression model coefficients for estimating derived
20-minute and 60-minute concentrations of systemic 13C-CA
(IV) in the HepQuant cholate DuO liver function test.

| Coefficient | Description | Value |
|---|---|---|
| Standardization | | |
| constants | | |
| Z_{BW, mean} | Average body weight | 91.751005 |
| Z_{BW, SD} | SD of body weight | 23.621730 |
| Z_{BMI, mean} | Average body mass index | 32.043528 |
| Z_{BMI, SD} | SD of body mass index | 7.463782 |
| Z_{PO(20), mean} | Average of ln(13C-CA) at 20 | 0.198711 |
| | min | |
| Z_{PO(20), SD} | SD of ln(13C-CA) at 20 min | 0.943803 |
| Z_{PO(60), mean} | Average of ln(13C-CA) at 60 | −0.173990 |
| | min | |
| Z_{PO(60), SD} | SD of ln(13C-CA) at 60 min | 0.826406 |
| Z_{T20, mean} | Average of 20-min sample | 19.996920 |
| | times | |
| Z_{T20, SD} | SD of 20-min sample times | 0.876646 |
| 20-min. IV model | | |
| β_{0, 20} | Intercept | 0.553399 |
| β_{BW, 20} | Body weight | −0.090211 |
| β_{BMI, 20} | Body mass index | 0.039738 |
| β_{PO(20), 20} | Ln(d4-CA) at 20 min | 0.068113 |
| β_{PO(60), 20} | Ln(d4-CA) at 60 min | 0.340721 |
| β_{T20} | Time of 20-min sample | −0.029428 |
| 60-min. IV model | | |
| β_{0, 60} | Intercept | −0.600173 |
| β_{BW, 60} | Body weight | −0.015905 |
| β_{PO(20), 60} | Ln(d4-CA) at 20 min | 0.084136 |
| β_{PO(60), 60} | Ln(d4-CA) at 60 min | 0.473529 |

Area under the oral curve (AUC_{Oral}) is measured by first simulating the full oral clearance curve using a compartmental model and calculating the area using the trapezoidal numerical integration.

Portal hepatic filtration rate (HFR_{P}) is the portal clearance adjusted for subject body weight (BW) calculated by Equation 1.

$${HFR}_{P} = \frac{{D}_{PO}}{{AUC}_{Oral} \cdot BW} . \qquad Eqn . 1$$

The term “Systemic HFR”, (systemic hepatic filtration rate, HFRs) mL min⁻¹ kg⁻¹ may be used to Model independent clearance of intravenously injected 13C-cholate, adjusted for body weight, and calculated from dose/AUC. The “Systemic HFR”, mL mini⁻¹ kg⁻¹, may be used to Model independent clearance of intravenously injected 13C-cholate, adjusted for body weight, and calculated from dose/AUC.

Systemic hepatic filtration rate (HFR_{S}) is the estimated systemic clearance adjusted for body weight (Equation 2). Here, the IV dose (D_{IV}) is assumed to be half of the administered oral dose amount.

$${HFR}_{S} = \frac{{D}_{IV}}{{AUC}_{IV} \cdot BW} . \qquad Eqn . 2$$

The term “STAT test” (STAT) refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. In one specific aspect, the STAT test is a simplified convenient test intended for screening purposes that can reasonably estimate the portal blood flow (estimated flow rate) from a single blood sample taken 60 minutes after orally administered deuterated-cholate. In some embodiments, STAT, is the d4-cholate concentration in the 60 minute blood sample. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. STAT test value may be used to estimate portal HFR, as provided in U.S. Pat. Nos. 8,961,925, 10,222,366. A STAT test value in a patient may be used to estimate a DSI value in a patient, as provided herein.

The term “DSI test” (DSI) refers to liver function Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and can be calculated from both portal and systemic HFRs. Based on the reproducibility of DSI values, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety.

The term “RCA20” represents the amount of the intravenously administered distinguishable compound, for example, a distinguishable cholate compound such as 13C-CA, that remains in the circulation 20 minutes after the intravenous injection.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009-01-28) “MELD/PELD calculator documentation”, which is incorporated herein by reference. For example, in interpreting the MELD score in hospitalized patients, the 3 month mortality is: 71.3% mortality for a MELD score of 40 or more.

The term “standard sample” refers to a sample with a known concentration of an analyte used for comparative purposes when analyzing a sample containing an unknown concentration of analyte.

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Steatotic Liver Disease” (SLD) encompasses various etiologies of hepatic steatosis.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Metabolic dysfunction-Associated Steatohepatitis (MASH), formerly known as “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases the fat accumulation can be associated with inflammation and scarring in the liver. This more serious form of the disease is termed metabolic dysfunction-associated steatohepatitis (MASH), formerly known as non-alcoholic steatohepatitis (NASH). MASH is associated with a much higher risk of liver fibrosis and cirrhosis than MASLD. Patients with MASH have increased risk for hepatocellular carcinoma. MASLD may progress to MASH with fibrosis cirrhosis and hepatocellular carcinoma.

The term “Metabolic dysfunction-Associated Steatotic Liver Disease” (MASLD), formerly known as “Non-Alcoholic Fatty Liver Disease” (NAFLD) refers to a common chronic liver disease characterized in part by a fatty liver condition with associated risk factors of obesity, metabolic syndrome, and insulin resistance. Both MASLD and MASH are often associated with obesity, diabetes mellitus and asymptomatic elevations of serum ALT and gamma-GT. Ultrasound monitoring can suggest the presence of a fatty infiltration of the liver; differentiation between MASLD and MASH, typically requires a liver biopsy.

The term “Metabolic dysfunction-associated Alcoholic Liver Disease (Met-ALD), refers to MASLD patients that consume greater amounts of alcohol per week (>140 g/week females and >210 g/week males).

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulation, portal hypertension and portal-systemic shunting to varices-ascites and encephalopathy. Altered portal flow is an indication of clinical complications.

Any appropriate analysis method known in the art may be employed for quantification of the distinguishable cholate compounds in blood or serum samples. For example, detection and quantification of the distinguishable cholate compound in the sample may comprise high performance liquid chromatography (HPLC), HPLC-diode-array detection (HPLC-DAD), HPLC-fluorescence, ultra-performance liquid chromatography (UPLC), mass spectrometry (MS), gas chromatography-MS (GC-MS), LC-MS, LC-MS/MS, surface-enhanced Raman spectroscopy (SERS), or immunoassays, for example, using isolated antibodies, monoclonal antibodies, or antigen-binding fragments thereof, single domain antibodies, aptamers, and the like. Methods for detection and quantification of distinguishable cholates are described in, for example, US20210318274, which is incorporated herein by reference in its entirety.

The blood or serum sample for use in the present methods may be collected from a subject by any known method in the art. For example, see WHO guidelines on drawing blood: best practices in phlebotomy, World Health Organization, 2010, Geneva, Switzerland or BP-EIA: Collecting, processing, and handling venous, capillary, and blood spot samples, PATH, 2005. For example, venipuncture using needle and syringe or indwelling catheter, arterial blood sampling, pediatric or neonatal blood sampling, or capillary sampling may be employed. The choice of site and procedure may depend on the volume of blood needed for the procedure and laboratory test to be done. For example, a venous site, finger-prick or heel-prick, also known as capillary sampling or skin puncture, may be employed.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

A method is provided for prediction of risk for clinical outcome in patients with compensated advanced chronic liver disease (cACLD) based on an oral cholate challenge test (DuO liver function test).

### Prognostic Model Development Study and Retrospective Analysis

A retrospective analysis of the Quantitative Liver Function Test (QLFT) ancillary study of the Hepatitis C Antiviral Long-term Treatment Against Cirrhosis Trial (HALT-C) was performed, whose study design, subject eligibility, and primary results have been previously characterized [16, 17, 19, 20]. The HALT-C trial was registered at ClinicalTrials.gov (NCT00006164). The HALT-C QLFT study was conducted according to the Declarations of Helsinki and Istanbul. All participants provided written informed consent to participate. The study was approved by the respective institutional review boards of the participating centers (COMIRB).

The study included 277 patients who had analytical retesting of their samples by liquid chromatography mass spectrometry (LC-MS); 220 of whom entered the randomized phase for long-term follow-up for up to 7.9 years for clinical outcome, and 215 of whom were event-free at the time of baseline testing. The primary clinical outcome was a composite of either liver-related death, Child-Pugh (CP) progression, variceal hemorrhage, ascites, and/or encephalopathy.

Inclusion criteria for randomization were active chronic hepatitis C with positive HCV RNA despite peginterferon/ribavirin treatment, advanced fibrosis or cirrhosis on liver histology, lack of other confounding liver diseases, and likely ability to complete the study [20]. Patients with CP class B or C cirrhosis, hepatocellular carcinoma, history of variceal hemorrhage, ascites, encephalopathy, significant cardiac, pulmonary or renal disease, and patients on the transplant waiting list were excluded. Cholate challenge test parameters and test versions

Cholate challenge tests were administered to fasting subjects (overnight or >5 h) following the standard test procedure according to Everson et al., 2007, Aliment Pharmacol Ther. 2007; 26(3):401-10. Various cholate challenge test versions were then retrospectively computed from measurements of labeled cholate concentrations in blood samples. (McRae et al., Clin Transl Sci. 2024; 17(4):e13786; McRae et al., Basic Clin Pharmacol Toxicol. 2024; 134(3):385-95). This current study focused on the results with the DuO distinguishable cholate challenge test, which involves an oral administration of 40 mg [2,2,4,4-²H](d4) cholate followed by blood samples collected at 20 and 60 minutes. Serum concentrations of cholate isotopes were quantified using LC-MS, and the following test parameters were calculated.

The term “Disease Severity Index”, or (DSI) refers to a liver disease severity index score from 0 to 50 indexing a subject's cholate clearances against maximum clearances of healthy controls.

The term “Hepatic Reserve” refers to percentage of maximum hepatic functional capacity measured by DSI, indexed hepatic reserve may be normalized to the DSI range in subjects of lean body mass. HR (algebraic) is simply an algebraic conversion of the DSI value in the subject: HR [100−(2×DSI)]. Indexed HR is normalized against the results within a cohort of normal lean controls. Hepatic Reserve (%) refers to a hepatic liver reserve score ranging from 100% (normal reserve) to 0% (no reserve) and indexes a subject's cholate clearances against a lower limit of clearances of healthy controls of lean body mass.

The term “Hepatic filtration rates” (HFR) are cholate clearances adjusted for patient body weight. Portal HFR is the apparent clearance of orally administered distinguishable cholate, such as d4-cholate, and systemic HFR is the clearance of intravenously administered distinguishable cholate, such as 13C-cholate. In some cases systemic HFR is the estimated clearance of cholate from a linear regression model if the distinguishable cholate were administered intravenously, such as clearance of intravenously administered 13C-cholate [13]. In the oral-only DuO test version, the systemic clearance of 13C-cholate is derived rather than measured.

The term “SHUNT %” refers to the portal-systemic shunt fraction, which is defined as the ratio of the systemic and portal distinguishable cholate clearances.

The “RISK ACE” test scores estimate the probability of adverse clinical events at or before the index time and are calculated using DSI values in Cox proportional hazards regression. A plurality of RISK ACE models were developed. For example, the BASELINE model (Model A) evaluates the risk using the DSI results from a single HepQuant DuO test; the FOLLOW-UP model (Model D) evaluates the risk at follow-up using the baseline DSI and the rate of change in DSI from baseline and is intended for monitoring applications to track liver function over time.

The term “Cox regression” refers to a statistical model that predicts the probability of an event occurring at a given time by building a model based on time-to-event data. It is also known as proportional hazards regression.

Methods: Data from 215 chronic hepatitis C subjects with cACLD who enrolled in the Quantitative Liver Function Ancillary study of the HALT-C trial (ClinicalTrials.gov, NCT00006164) were analyzed retrospectively. Subjects were tested at baseline and 2 years and followed prospectively for clinical events (liver-related death, Child-Pugh progression, variceal hemorrhage, ascites, and/or encephalopathy) for up to 7.9 years. Cox proportional hazards regression models were developed to predict risk for adverse clinical events (RISK ACE) using the disease severity index (DSI) from the oral distinguishable cholate challenge.

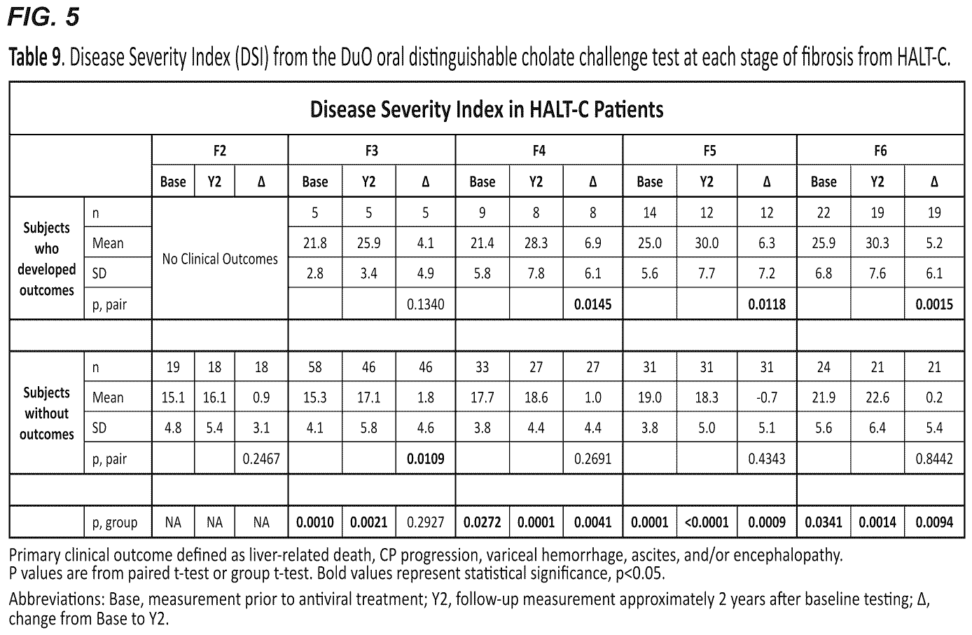

FIG. 5 shows Table 9 with Disease Severity Index (DSI) values from the DuO oral distinguishable cholate challenge test at each stage of fibrosis (F2 to F6) from HALT-C patients who developed clinical outcomes and subjects without clinical outcomes. Primary clinical outcome was defined as liver-related death, CP progression, variceal hemorrhage, ascites, and/or encephalopathy. P values are from paired t-test or group t-test. Bold values represent statistical significance, p<0.05. abbreviations: Base, measurement to antiviral treatment; Y2, follow-up measurement approximately 2 years after baseline testing; D, change from base to Y2.

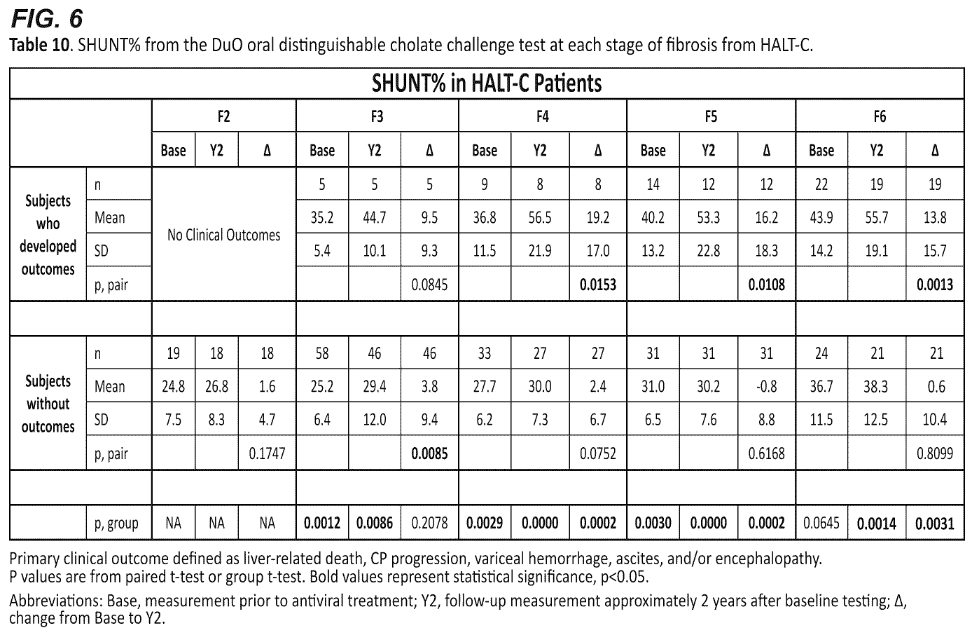

FIG. 6 shows Table 10 with SHUNT % from the DuO oral distinguishable cholate challenge test at each stage of fibrosis from HALT-C patients who developed clinical outcomes and subjects without clinical outcomes. Primary clinical outcome was defined as liver-related death, CP progression, variceal hemorrhage, ascites, and/or encephalopathy. P values are from paired t-test or group t-test. Bold values represent statistical significance, p<0.05. abbreviations: Base, measurement to antiviral treatment; Y2, follow-up measurement approximately 2 years after baseline testing; D, change from base to Y2.

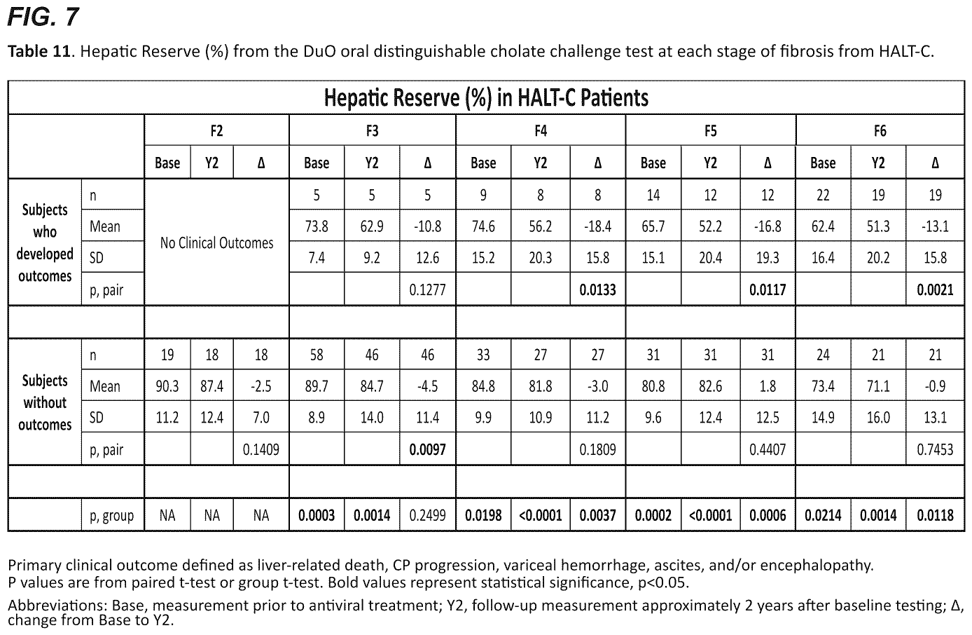

FIG. 7 shows Table 11 with Hepatic Reserve (%) from the DuO oral distinguishable cholate challenge test at each stage of fibrosis from HALT-C patients who developed clinical outcomes and subjects without clinical outcomes. Primary clinical outcome was defined as liver-related death, CP progression, variceal hemorrhage, ascites, and/or encephalopathy. P values are from paired t-test or group t-test. Bold values represent statistical significance, p<0.05. abbreviations: Base, measurement to antiviral treatment; Y2, follow-up measurement approximately 2 years after baseline testing; D, change from base to Y2.

### Statistical Analysis

Continuous variables were reported as mean±standard deviation or number (percentage). Differences between subgroups of DSI (above or below 18.3) at baseline were analyzed for significance using t test for continuous data and by chi-squared test for proportions [21, 22]. In all analyses, statistical significance was set at p<0.05. Univariate hazard ratios for baseline predictors of survival corresponding to the primary clinical outcome (liver-related death, CP progression, variceal hemorrhage, ascites, and/or encephalopathy) were sorted by Chi-square statistic.

The RISK ACE scores were calculated using the predicted risk from Cox proportional hazards regression. Baseline risk was estimated from the baseline hazard using standard methods as implemented in the survival package in the R statistical language [23].

Time was measured from the date that the distinguishable cholate challenge test was administered until the subject experienced an outcome event or was event-free (censored) at their last follow-up visit. The RISK ACE score is the predicted risk for the occurrence of an adverse clinical event (liver-related death, CP progression, variceal hemorrhage, ascites, and/or encephalopathy) before an index time in the future (i.e., one minus the predicted event-free survival probability at an index time). Four RISK ACE model variants were developed.
Model A evaluates risk using baseline DSI as the only predictor. Model A is also known as the BASELINE model.
Model B evaluates risk at follow-up and included DSI at baseline and subsequent change in DSI after approximately two years as predictors.
Model C evaluates risk at follow-up and included DSI at baseline and subsequent DSI after approximately two years as predictors.
Model D evaluates risk at follow-up and included DSI at baseline and the rate of change from baseline (i.e., change in DSI divided by the time in months between the baseline and follow-up test) as predictors. Models B, C and D are follow-up models. Model D is also known as the FOLLOW UP model.
Model A included all subjects that had baseline DSI measurements. Models B, C, and D included only those subjects that had both baseline and Year 2 DSI measurements and only subjects that had not experienced events before the Year 2 test date.

Predicted risk by the baseline model (Model A) was evaluated at various quartiles of DSI and compared to the Kaplan-Meier estimated risk. Predicted risk by all models over 5 years in 6-month increments were compared to actual risk as estimated by Kaplan-Meier. To explore the relationships of DSI at baseline and follow-up, the calculated RISK ACE (Model D) was evaluated for baseline DSI values of 18.3, 23, 30, and 40, with various changes in DSI after 2 years follow-up. The relationships between DSI, fibrosis stage, and clinical events were evaluated by Cox proportional hazards regression.

### Results

### Subject Characteristics, Laboratory Values, and Clinical Scores

The subject characteristics, laboratory values, and clinical scores at baseline are shown in Table 1B for the 215 in long-term follow-up.

TABLE 1B
Baseline subject characteristics, laboratory values, and clinical scores, as well as clinical outcomes observed
within the study period, in all subjects and by subgroups with low (≤18.3) and high (>18.3) DSI.

| | All Subjects | | DSI ≤18.3 | | DSI >18.3 | | |
|---|---|---|---|---|---|---|---|
| | | Mean ± | | Mean ± | | Mean ± | |
| | | SD or | | SD or | | SD or | |
| | n | n (%) | n | n (%) | n | n (%) | p value ^{a} |
| Age (years) | 215 | 49.93 ± 7.27 | 106 | 48.78 ± 6.65 | 109 | 51.05 ± 7.70 | 0.0222 |
| Weight (kg) | 215 | 89.39 ± 16.37 | 106 | 86.73 ± 16.53 | 109 | 91.98 ± 15.86 | 0.0184 |
| BMI (kg/m²) | 215 | 29.53 ± 4.86 | 106 | 28.04 ± 3.93 | 109 | 30.98 ± 5.25 | <0.0001 |
| Overweight (≥25 | 215 | 183 (85.1%) | 106 | 87 (82.1%) | 109 | 96 (88.1%) | 0.2173 |
| kg/m²) | | | | | | | |
| Obese (≥30 | 215 | 91 (42.3%) | 106 | 29 (27.4%) | 109 | 62 (56.9%) | <0.0001 |
| kg/m²) | | | | | | | |
| Male | 215 | 162 (75.3%) | 106 | 85 (80.2%) | 109 | 77 (70.6%) | 0.1034 |
| Race | | | | | | | |
| White | 215 | 160 (74.4%) | 106 | 87 (82.1%) | 109 | 73 (67.0%) | 0.0113 |
| Black | 215 | 26 (12.1%) | 106 | 10 (9.4%) | 109 | 16 (14.7%) | 0.2344 |
| Other | 215 | 29 (13.5%) | 106 | 9 (8.5%) | 109 | 20 (18.3%) | 0.0358 |
| Ethnicity | | | | | | | |
| Hispanic | 215 | 25 (11.6%) | 106 | 7 (6.6%) | 109 | 18 (16.5%) | 0.0239 |
| Non-Hispanic | 215 | 190 (88.4%) | 106 | 99 (93.4%) | 109 | 91 (83.5%) | — |
| Lab. values and | | | | | | | |
| clinical scores | | | | | | | |
| Bilirubin | 215 | 0.76 ± 0.40 | 106 | 0.69 ± 0.33 | 109 | 0.83 ± 0.45 | 0.0098 |
| (mg/dL) | | | | | | | |
| Albumin (g/dL) | 215 | 3.78 ± 0.39 | 106 | 3.95 ± 0.33 | 109 | 3.62 ± 0.38 | <0.0001 |
| Prothrombin | 215 | 1.02 ± 0.10 | 106 | 0.99 ± 0.08 | 109 | 1.05 ± 0.11 | <0.0001 |
| time, INR | | | | | | | |
| Platelets (×10³ | 215 | 163.86 ± 67.70 | 106 | 188.50 ± 61.09 | 109 | 139.89 ± 65.39 | <0.0001 |
| μL⁻¹) | | | | | | | |
| AST (U/L) | 215 | 88.25 ± 62.34 | 106 | 73.28 ± 53.07 | 109 | 102.80 ± 67.30 | 0.0005 |
| ALT (U/L) | 215 | 109.34 ± 85.84 | 106 | 103.96 ± 74.00 | 109 | 114.57 ± 96.03 | 0.3663 |
| Alkaline | 215 | 100.99 ± 41.40 | 106 | 84.17 ± 26.51 | 109 | 117.35 ± 46.53 | <0.0001 |
| phosphatase (U/L) | | | | | | | |
| Spleen volume | 208 | 454 ± 294 | 101 | 370 ± 243 | 107 | 533 ± 316 | <0.0001 |
| (mL) | | | | | | | |
| Liver volume | 211 | 1639 ± 361 | 103 | 1603 ± 353 | 108 | 1674 ± 369 | 0.1506 |
| (mL) | | | | | | | |
| CP score | 215 | 5.28 ± 0.46 | 106 | 5.12 ± 0.33 | 109 | 5.43 ± 0.52 | <0.0001 |
| MELD score | 215 | 6.87 ± 1.30 | 106 | 6.58 ± 1.03 | 109 | 7.17 ± 1.46 | 0.0008 |
| Clinical outcomes | | | | | | | |
| Primary clinical | 215 | 50 (23.3%) | 106 | 7 (6.6%) | 109 | 43 (39.4%) | <0.0001 |
| outcome ^{b} | | | | | | | |
| All cause death | 215 | 32 (14.9%) | 106 | 7 (6.6%) | 109 | 25 (22.9%) | 0.0008 |
| Liver-related | 215 | 17 (7.9%) | 106 | 1 (0.9%) | 109 | 16 (14.7%) | 0.0002 |
| death | | | | | | | |
| HCC | 215 | 6 (2.8%) | 106 | 1 (0.9%) | 109 | 5 (4.6%) | 0.0995 |
| CP progression, | 215 | 40 (18.6%) | 106 | 6 (5.7%) | 109 | 34 (31.2%) | <0.0001 |
| CP ≥7 | | | | | | | |
| Variceal | 215 | 5 (2.3%) | 106 | 0 (0.0%) | 109 | 5 (4.6%) | 0.0258 |
| hemorrhage | | | | | | | |
| Ascites | 215 | 18 (8.4%) | 106 | 2 (1.9%) | 109 | 16 (14.7%) | 0.0007 |
| Encephalopathy | 215 | 10 (4.7%) | 106 | 2 (1.9%) | 109 | 8 (7.3%) | 0.0604 |
| Liver transplant | 215 | 8 (3.7%) | 106 | 1 (0.9%) | 109 | 7 (6.4%) | 0.0328 |

^{a} Differences between DSI ≤18.3 and DSI >18.3 groups; continuous variables by t test and categorical predictors by Chi-squared test for proportions. Bold values represent statistical significance, p < 0.05.
^{b} Primary clinical outcome defined as liver-related death, CP progression, variceal hemorrhage, ascites, and/or encephalopathy.

Abbreviations: ALT, alanine transaminase; AST, aspartate transaminase; BMI, body mass index; CP, Child Pugh; DSI, disease severity index; HCC, hepatocellular carcinoma; INR, international normalized ratio; MELD, Model for End-Stage Liver Disease.

The mean age was 50±7 years, 75% were male, mean BMI was 30±5 kg/m², 74% were White, 12% were Black, and 12% were Hispanic. Eighty-five percent of the subjects were overweight and 42% were obese. Mean albumin was 3.78±0.39 g/dL, alkaline phosphatase 100.99±41.40 U/L, ALT 109.34±85.84 U/L, AST 88.25±62.34 U/L, bilirubin 0.76±0.40 mg/dL, international normalized ratio (INR) 1.02±0.10, and platelet count 164,000±68,000 μL⁻¹. These results and the clinical scores (Model for End-Stage Liver Disease [MELD]6.87±1.30, CP 5.28±0.46) suggested that these subjects with advanced fibrosis or compensated (Child-Pugh A) cirrhosis were well-compensated. However, the results also revealed that these tests were insensitive in the detection of significant disease—the means for albumin, bilirubin, and INR were within the normal range.

Of the 215 subjects, 50 experienced decompensation events: 17 experienced liver-related death, 40 experienced a progression in CP score of at least two points, 5 had variceal hemorrhage, 18 developed ascites, 10 experienced encephalopathy, and 8 had liver transplantation. Of the 40 subjects experiencing CP progression, 23 (56%) also experienced one or more the hard primary clinical outcomes (liver-related death, variceal hemorrhage, ascites, and/or encephalopathy).

When stratified by liver function (baseline DuO distinguishable cholate challenge test values) (Table 1B), subjects with high baseline DSI (i.e., above the 18.3 cutoff for ruling out large esophageal varices [15]) had significantly worse laboratory values and clinical scores, except for ALT (p=0.3663) and liver volume (p=0.1506). Forty-three (40%) subjects with baseline DSI>18.3 experienced the primary clinical outcome compared to 7 (7%) subjects with baseline DSI≤18.3 (p<0.0001).

### DuO Distinguishable Cholate Test Results

The DuO test results are shown in Table 2 for all subjects and by subgroup that experienced or did not experience primary clinical outcome. In the subgroup that did not experience clinical outcome, the mean values for test parameters at baseline met the thresholds for ruling out large esophageal varices (DSI≤18.3, SHUNT %≤30.0%, and Hepatic Reserve ≥83.4%). In the subgroup that experienced clinical outcome, the mean values for test parameters at baseline exceeded the same thresholds. Test parameters at Year 2 were slightly worse in those without outcomes and substantially worse in those with outcomes. All test parameters, at baseline and Year 2, were significantly worse (i.e., higher DSI and SHUNT %; lower Hepatic Reserve, portal HFR, and systemic HFR) in subjects who experienced a clinical outcome (p)10<0.0).

TABLE 2
DuO distinguishable cholate challenge test results for all subjects and by subjects who
either did or did not experience the primary clinical outcome within the study period.

| | | | No clinical | | | | |
|---|---|---|---|---|---|---|---|
| | All subjects | | outcome | | Clinical outcome ^{a} | | |
| Test Parameter | n | Mean ± SD | n | Mean ± SD | n | Mean ± SD | p value ^{b} |
| Baseline | | | | | | | |
| DSI | 215 | 19.03 ± 5.99 | 165 | 17.40 ± 4.90 | 50 | 24.41 ± 6.15 | <0.0001 |
| SHUNT % (%) | 215 | 31.28 ± 10.95 | 165 | 28.42 ± 8.43 | 50 | 40.72 ± 12.94 | <0.0001 |
| Hepatic Reserve (%) | 215 | 80.55 ± 14.95 | 165 | 84.75 ± 11.93 | 50 | 66.66 ± 15.59 | <0.0001 |
| Portal HFR (mL/min/kg) | 215 | 14.04 ± 7.22 | 165 | 15.57 ± 6.96 | 50 | 9.00 ± 5.60 | <0.0001 |
| Systemic HFR | 215 | 3.74 ± 0.77 | 165 | 3.94 ± 0.67 | 50 | 3.09 ± 0.70 | <0.0001 |
| (mL/min/kg) | | | | | | | |
| Year 2 ^{c} | | | | | | | |
| DSI | 187 | 20.94 ± 7.69 | 143 | 18.34 ± 5.72 | 44 | 29.36 ± 7.27 | <0.0001 |
| SHUNT % (%) | 187 | 36.14 ± 16.42 | 143 | 30.67 ± 10.42 | 44 | 53.92 ± 19.61 | <0.0001 |
| Hepatic Reserve (%) | 187 | 75.39 ± 19.45 | 143 | 82.05 ± 13.96 | 44 | 53.77 ± 19.14 | <0.0001 |
| Portal HFR (mL/min/kg) | 187 | 12.60 ± 7.39 | 143 | 14.61 ± 7.07 | 44 | 6.09 ± 3.77 | <0.0001 |
| Systemic HFR | 187 | 3.59 ± 0.92 | 143 | 3.87 ± 0.78 | 44 | 2.66 ± 0.72 | <0.0001 |
| (mL/min/kg) | | | | | | | |

^{a} Primary clinical outcome defined as liver-related death, CP progression, variceal hemorrhage, ascites, and/or encephalopathy.
^{b} Differences between subjects who did/did not experience the primary clinical outcome by t test. Bold values represent statistical significance, p < 0.05.
^{c} Actual time intervals were calculated from the dates of testing. Includes subjects who experienced clinical outcome between baseline and Year 2.
Abbreviations: DSI, disease severity index; HFR, hepatic filtration rate; SHUNT %, portal-systemic shunting.

### Derivation of RISK ACE Models

Univariate baseline predictors of the time to the first adverse clinical event are shown in Table 3. The strongest predictors were Hepatic Reserve (χ²=64) and DSI (χ²=61). A 1-unit increase in baseline Hepatic Reserve was associated with a 6% reduction in risk, and a 1-unit increase in DSI was predictive of an 18% increase in event risk. Other measures of liver function (SHUNT % χ²=58 and portal HFR χ²=38) were also among the strongest predictors of clinical events. Other strong predictors included spleen volume, CP score, albumin, and Ishak fibrosis score.

TABLE 3
Univariate hazard ratio (HR) analysis for baseline predictors
of survival corresponding to the primary clinical outcome (liver-
related death, CP progression, variceal hemorrhage, ascites,
and/or encephalopathy) sorted by Chi-square statistic.

| Predictor | HR (95% CI) | | χ^{2 a} | Pr(>\|χ²]) ^{a} |
|---|---|---|---|---|
| Hepatic Reserve (%) | 0.94 | (0.92-0.95) | 63.51 | <0.0001 |
| DSI | 1.18 | (1.13-1.23) | 61.31 | <0.0001 |
| SHUNT % (%) | 1.08 | (1.06-1.10) | 57.92 | <0.0001 |
| Spleen volume (mL) | 1.00 | (1.00-1.00) | 56.82 | <0.0001 |
| Albumin (g/dL) | 0.10 | (0.05-0.20) | 43.33 | <0.0001 |
| CP score | 6.67 | (3.78-11.76) | 42.96 | <0.0001 |
| Portal HFR (mL/min/kg) | 0.80 | (0.75-0.86) | 37.81 | <0.0001 |
| Fibrosis (Ishak) | 1.99 | (1.54-2.57) | 28.08 | <0.0001 |
| Prothrombin time, INR | 86.11 | (14.28-519.31) | 23.62 | <0.0001 |
| Platelet count (×10³ μL⁻¹) | 0.99 | (0.98-0.99) | 23.19 | <0.0001 |
| Bilirubin (mg/dL) | 4.12 | (2.31-7.35) | 23.04 | <0.0001 |
| Fibrosis (METAVIR) | 3.29 | (2.02-5.35) | 22.9 | <0.0001 |
| Alkaline phosphatase (U/L) | 2.57 | (1.73-3.82) | 21.98 | <0.0001 |
| AST (U/L) | 1.01 | (1.01-1.02) | 17.81 | <0.0001 |
| BMI (kg/m²) | 1.00 | (1.00-1.01) | 6.1 | 0.0135 |
| Liver volume (mL) | 1.07 | (1.01-1.13) | 5.66 | 0.0174 |
| Race (white = 1, all others = | 1.00 | (1.00-1.00) | 1.54 | 0.2152 |
| 0) | | | | |
| ALT (U/L) | 0.74 | (0.41-1.34) | 0.97 | 0.3253 |
| Age (years) | 1.00 | (0.99-1.00) | 0.55 | 0.4566 |
| Sex (male = 1, female = 0) | 0.99 | (0.95-1.03) | 0.26 | 0.6075 |

^{a} Chi-square statistic and corresponding p value for the univariate significance of the association of the variable with the event hazard. Bold values represent statistical significance, p < 0.05.
Abbreviations: ALT, alanine transaminase; AST, aspartate transaminase; BMI, body mass index; CI, confidence interval; CP, Child Pugh; DSI, disease severity index; HFR, hepatic filtration rate; HR, hazard ratio; INR, international normalized ratio; SHUNT %, portal-systemic shunting.

RISK ACE model predictors were selected based on the results of Table 3 which found that the liver function measures were the strongest predictors. DSI, SHUNT %, and Hepatic Reserve are all derived from the same cholate clearance and are colinear. Collinearity of predictor variables in regression models can lead to model instability (inflated variance and inability of the models to estimate the independent effects of the colinear variables). Therefore, a single function measure (DSI) was selected in RISK ACE given its use in a wider range of clinical studies and as a validated parameter in ruling out large esophageal varices [15]. Table 4 shows the coefficients for the RISK ACE models. The overall fit by likelihood ratio for all models were highly significant (p<0.0001).

TABLE 4
RISK ACE coefficients from Cox proportional hazards regression.

| Model/Variable | β | HR (95% CI) | | Std. Error | z | Pr(>\|z\|) |
|---|---|---|---|---|---|---|
| Model A ^{a} | | | | | | |
| Baseline DSI | 0.167 | 1.182 | (1.134-1.233) | 0.021 | 7.830 | <0.0001 |
| Model B ^{b} | | | | | | |
| Baseline DSI | 0.219 | 1.245 | (1.153-1.344) | 0.039 | 5.596 | <0.0001 |
| 2-year change from baseline DSI | 0.137 | 1.147 | (1.067-1.233) | 0.037 | 3.706 | 0.0002 |
| Model C ^{b} | | | | | | |
| Baseline DSI | 0.082 | 1.086 | (0.999-1.18) | 0.042 | 1.936 | 0.0529 |
| 2-year DSI | 0.137 | 1.147 | (1.067-1.233) | 0.037 | 3.706 | 0.0002 |
| Model D ^{b} | | | | | | |
| Baseline DSI | 0.209 | 1.233 | (1.145-1.328) | 0.038 | 5.526 | <0.0001 |
| Rate of change from baseline DSI | 2.669 | 14.432 | (3.026-68.821) | 0.797 | 3.349 | 0.0008 |

^{a} 50 clinical events were experienced in 215 subjects who were event-free at baseline
^{b} 23 clinical events were experienced in 160 subjects who were event-free at Year 2
Bold values represent statistical significance, p < 0.05.
Abbreviations: β, regression coefficient; DSI, disease severity index; HR, hazard ratio; RISK ACE, risk for adverse clinical events models.

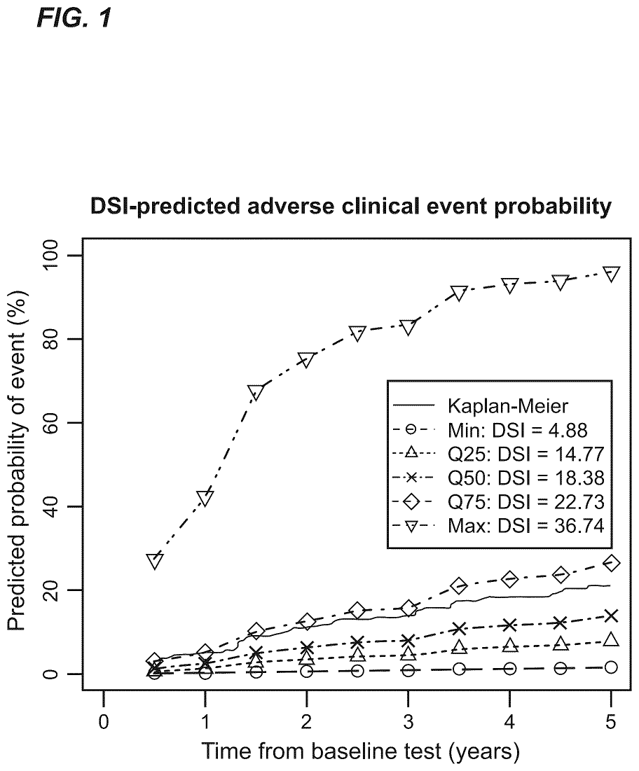

Model A evaluates the risk using baseline DSI as the only predictor. For example, FIG. 1 shows the predicted adverse clinical event probability by RISK ACE (Model A) by quartiles of DSI, demonstrating a nonlinear relationship between risk and baseline DSI. Model A is intended to be used in interpreting the results of a single DuO distinguishable cholate challenge test (e.g., a patient's first test).

As with any disease, risk may be not only a function of baseline condition, but also may be determined by the rate of disease progression. Therefore, additional RISK ACE models were considered that included both baseline and follow-up DSI measurements. Models B, C, and D estimate the risk using baseline and follow-up DSI measurements as predictors (Model B, two-year change from baseline; Model C, DSI at two years; Model D, rate of change from baseline) (Table 4). In general, the Models' hazard ratios show that higher DSI at baseline was associated with higher risk of adverse clinical events. Increasing DSI or increasing rate of DSI change at follow-up was associated with significantly higher risk of adverse clinical events (p<0.001). For Model C, the DSI measurements at two years (HR 1.15 [95% CI: 1.07-1.23], p=0.0002) had a closer association with risk than DSI measured at baseline (HR 1.09 [95% CI: 1.00-1.18], p=0.0529). Since Models B, C, and D require results from more than one DuO distinguishable cholate challenge test, these models are intended for monitoring applications to track liver function over time.

Table 5 compares the predicted risk from RISK ACE models to the actual risk from the Kaplan-Meier curve over a period of 5 years at 6-month intervals following the baseline test. There were no substantive differences between the actual and predicted risks. Models B and C were identical in terms of the risk predictions because the information contained in the predictors was the same (i.e., including baseline DSI and change from baseline in the proportional hazards model is the same as including baseline DSI and follow-up DSI).

TABLE 5
Predicted risk of adverse clinical events (RISK ACE) over 5 years.

| Years from | | Predicted risk of an event | | | Difference from actual (predicted | | |
|---|---|---|---|---|---|---|---|
| baseline or | | before indicated times (%) | | | minus actual risk of event) (%) | | |
| Year 2 | Actual | | Models B & | | | Models B & | |
| test | (%) ^{a} | Model A | C ^{b} | Model D | Model A | C ^{b} | Model D |

Baseline RISK ACE

| | | | | | | | |
|---|---|---|---|---|---|---|---|
| 0.5 | 2.80 | 2.83 | — | — | 0.03 | — | — |
| 1.0 | 4.71 | 4.72 | — | — | 0.02 | — | — |
| 1.5 | 9.06 | 8.98 | — | — | −0.08 | — | — |
| 2.0 | 11.02 | 10.85 | — | — | −0.18 | — | — |
| 2.5 | 13.02 | 12.75 | — | — | −0.27 | — | — |
| 3.0 | 13.54 | 13.26 | — | — | −0.28 | — | — |
| 3.5 | 17.32 | 17.08 | — | — | −0.24 | — | — |
| 4.0 | 18.44 | 18.25 | — | — | −0.19 | — | — |
| 4.5 | 19.10 | 18.91 | — | — | −0.19 | — | — |
| 5.0 | 21.13 | 20.94 | — | — | −0.19 | — | — |

Follow-up RISK ACE

| | | | | | | | |
|---|---|---|---|---|---|---|---|
| 0.5 | 0.63 | — | 0.61 | 0.61 | — | −0.02 | −0.02 |
| 1.0 | 2.63 | — | 2.54 | 2.57 | — | −0.10 | −0.07 |
| 1.5 | 7.59 | — | 7.67 | 7.75 | — | 0.08 | 0.16 |
| 2.0 | 7.59 | — | 7.67 | 7.75 | — | 0.08 | 0.16 |
| 2.5 | 11.00 | — | 11.09 | 11.12 | — | 0.10 | 0.13 |
| 3.0 | 11.87 | — | 11.96 | 11.97 | — | 0.09 | 0.10 |
| 3.5 | 15.73 | — | 15.36 | 15.32 | — | −0.37 | −0.41 |
| 4.0 | 16.82 | — | 16.22 | 16.17 | — | −0.61 | −0.65 |
| 4.5 | 18.23 | — | 17.13 | 17.10 | — | −1.10 | −1.13 |
| 5.0 | 20.18 | — | 18.23 | 18.20 | — | −1.94 | −1.98 |

^{a} Actual risk is calculated from the Kaplan-Meier curve
^{b} Models B and C yield the same results
Abbreviations: DSI, disease severity index; Model A, baseline DSI; Model B, baseline DSI and change from baseline DSI; Model C, baseline DSI and 2-year DSI; Model D, baseline DSI and rate of change in DSI.

### Evaluation of Risk at Baseline and Follow-Up

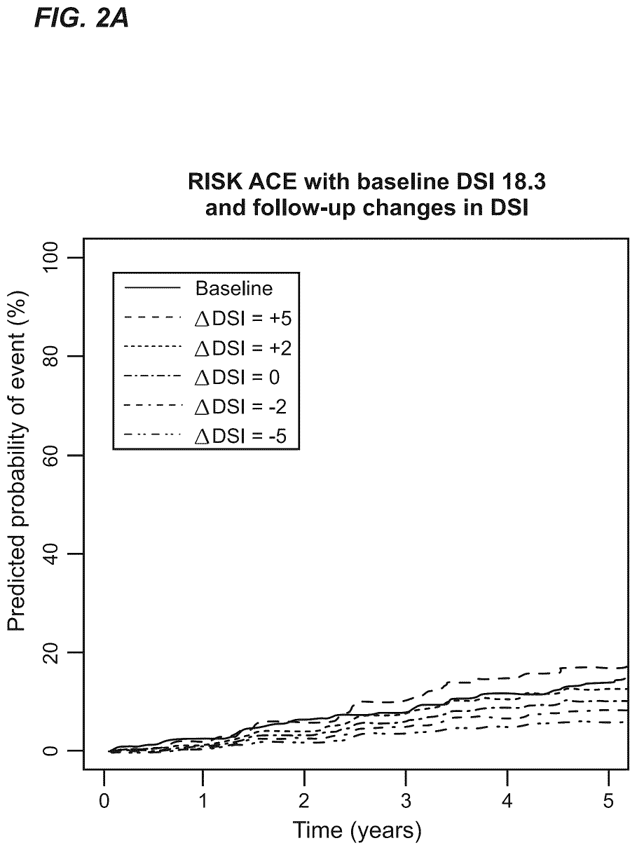

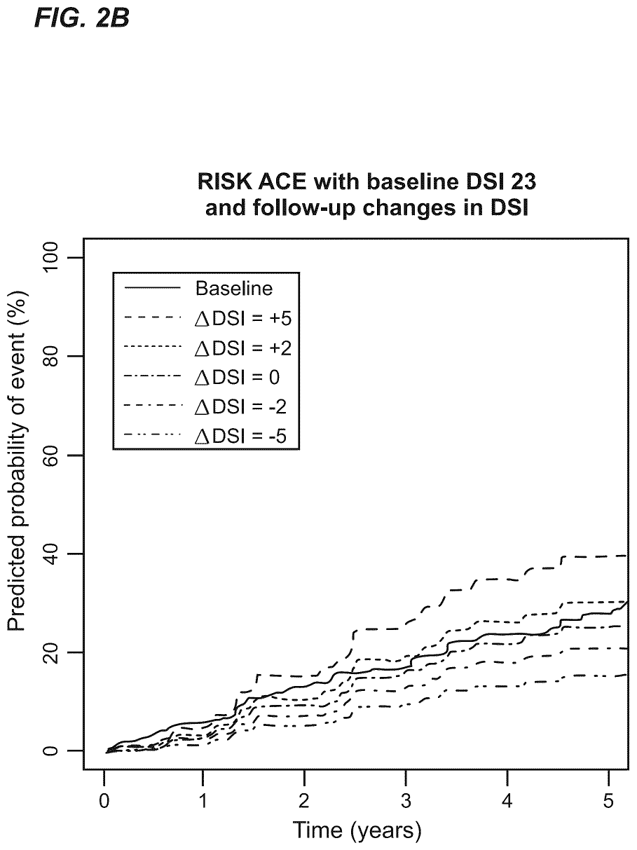

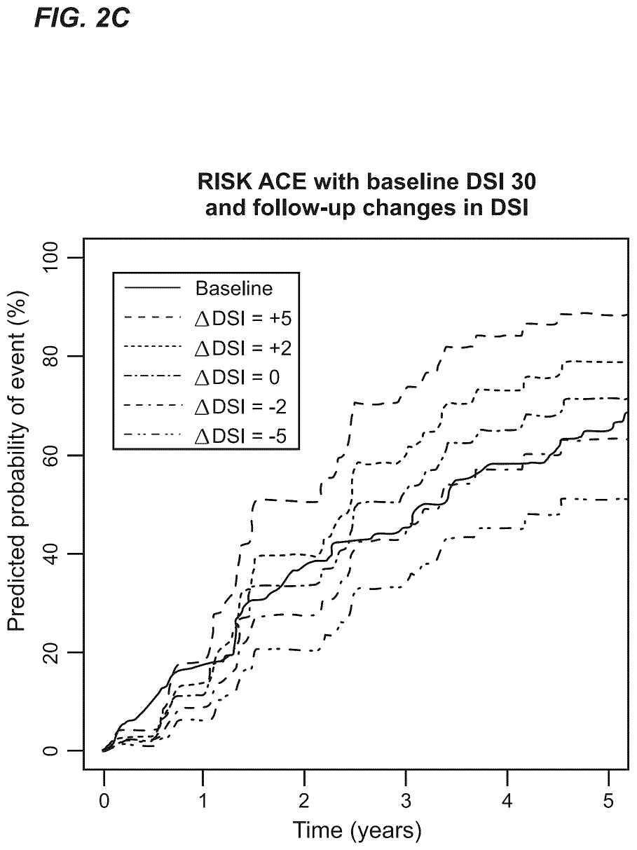

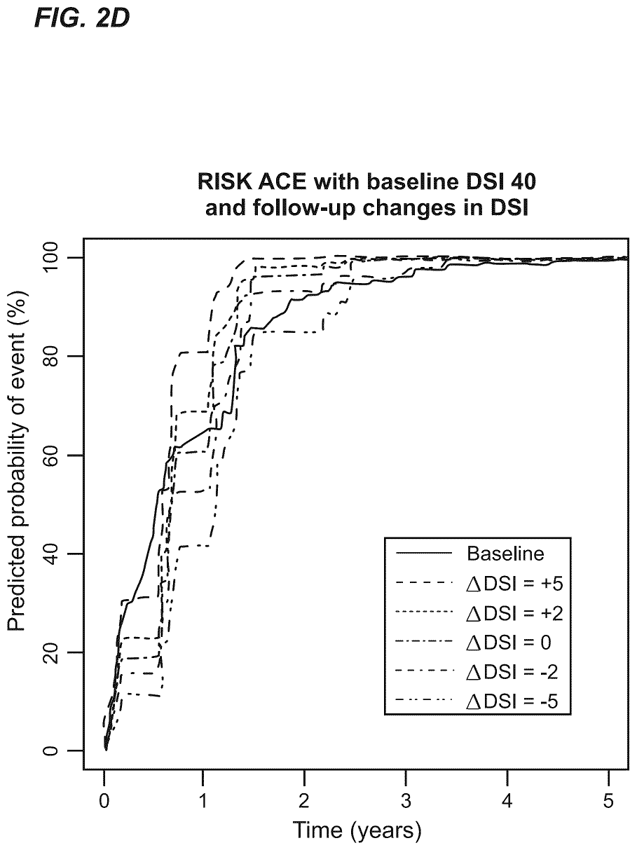

The relationship between RISK ACE and DSI at baseline and follow-up for baseline DSI values of DSI 18.3 (FIG. 2A), DSI 23 (FIG. 2B), DSI 30 (FIG. 2C), and DSI 40 (FIG. 2D) is illustrated in FIG. 2A-D. Previous analysis of the cross-sectional diagnostic accuracy of DSI (mixed etiologies with CP A cirrhosis) demonstrated that DSI 18.3 was a moderate risk level associated with 4.7% probability of having large esophageal varices, and DSI 23 was a high-risk level associated with >9.4% probability of having large esophageal varices [15]. Applying Model A at these baseline DSI values yielded 6%, 13%, 37%, and 91% predicted risk for adverse clinical events at two years for baseline DSI values of 18.3, 23, 30, and 40 respectively (FIG. 2A-D, solid black lines).

RISK ACE evaluated using Model D (FIG. 2A-D, dashed lines) demonstrates a protective effect for stability in DSI (i.e., the estimated risk decreases when DSI stays the same over time), but only for those with lower DSI at baseline. For example, to yield similar 2-year risk predictions to Model A, the change in DSI (follow-up minus baseline) could be as high as +5 and +4 over two years for baseline DSI values 18.3 and 23. For higher baseline DSI values, the protective effect of stability in DSI diminishes. To obtain similar 2-year predictions to Model A, the change in DSI (follow-up minus baseline) would need to be +1 and −2 over two years for baseline DSI of 30 and 40, respectively. These results also suggest that progression of liver disease, as measured by an increase in the rate of change of DSI, has a greater effect on clinical outcome risk in those with higher baseline DSI.

### Relating DSI and Fibrosis to Risk for Clinical Outcome

The results of the Cox proportional hazards regression examining the relationship of DSI, fibrosis stage, and clinical outcome are shown in Table 6. At baseline, DSI (HR 1.15 [95% CI: 1.09-1.21], p<0.0001) and Ishak fibrosis stage (HR 1.34 [95% CI: 1.00-1.80], p=0.0467) were significant predictors for risk of future clinical events. At follow-up, baseline DSI (HR 1.21 [95% CI: 1.11-1.32], p<0.0001) and the rate of change in DSI (HR 16.87 [95% CI: 3.23-88.30], p=0.0008) were significant predictors, but not Ishak fibrosis stage (HR 1.20 [95% CI: 0.82-1.76], p=0.3458). These results suggest that functional testing by the DuO distinguishable cholate challenge test may be more sensitive than fibrosis staging at predicting risk for future clinical events.

TABLE 6
Results of Cox proportional hazards regression with DSI and Ishak fibrosis stage.

| | | | | | |
|---|---|---|---|---|---|
| Model/Variable | β | HR (95% CI) | Std. Error | z | Pr(>\|z\|) |

Fibrosis + Baseline DSI ^{a}

| | | | | | | |
|---|---|---|---|---|---|---|
| Fibrosis (Ishak) | 0.296 | 1.344 | (1.004-1.799) | 0.149 | 1.989 | 0.0467 |
| Baseline DSI | 0.136 | 1.146 | (1.088-1.206) | 0.026 | 5.201 | <0.0001 |

Fibrosis + Baseline DSI + Rate of Change in DSI ^{b}

| | | | | | | |
|---|---|---|---|---|---|---|
| Fibrosis (Ishak) | 0.184 | 1.202 | (0.82-1.761) | 0.195 | 0.943 | 0.3458 |
| Baseline DSI | 0.190 | 1.209 | (1.111-1.315) | 0.043 | 4.422 | <0.0001 |
| Rate of change in DSI | 2.826 | 16.874 | (3.225-88.300) | 0.844 | 3.347 | 0.0008 |

^{a} 50 clinical events were experienced in 215 subjects who were event-free at baseline
^{b} 23 clinical events were experienced in 160 subjects who were event-free at Year 2
Bold values represent statistical significance, p < 0.05.
Abbreviations: DSI, disease severity index; β, regression coefficient; HR, hazard ratio.

Table 7 shows baseline cholate SHUNT test results for all subjects and by subjects who either did or did not experience the primary clinical outcome within the study period. Primary clinical outcomes include liver-related death, CP progression, variceal hemorrhage, ascites, and/or encephalopathy. Cholate SHUNT test results were used to calculate DSI, SHUNT % (%)(portal-systemic shunting), Hepatic Reserve (%), portal HFR (mL/min/kg), or systemic HFR (mL/min/kg), from cholate SHUNT V1.0, SHUNT V1.1, or SHUNT V2.0.

TABLE 7
Cholate SHUNT test results

| | | | No clinical | | | | |
|---|---|---|---|---|---|---|---|
| Test Version/ | All subjects | | outcome | | Clinical outcome ^{a} | | |
| Parameter | n | Mean ± SD | n | Mean ± SD | n | Mean ± SD | p value ^{b} |
| SHUNT V1.0 | | | | | | | |
| DSI | 215 | 19.40 ± 5.66 | 165 | 17.82 ± 4.36 | 50 | 24.63 ± 6.34 | <0.0001 |
| SHUNT % (%) | 215 | 37.73 ± 15.31 | 165 | 34.25 ± 12.84 | 50 | 49.19 ± 17.23 | <0.0001 |
| Hepatic Reserve (%) | 215 | 79.44 ± 14.29 | 165 | 83.57 ± 10.84 | 50 | 65.83 ± 15.86 | <0.0001 |
| Portal HFR (mL/min/kg) | 215 | 13.16 ± 6.04 | 165 | 14.58 ± 5.64 | 50 | 8.48 ± 4.89 | <0.0001 |
| Systemic HFR | 215 | 4.25 ± 1.25 | 165 | 4.46 ± 1.22 | 50 | 3.57 ± 1.14 | <0.0001 |
| (mL/min/kg) | | | | | | | |
| SHUNT V1.1 | | | | | | | |
| DSI | 215 | 19.17 ± 5.68 | 165 | 17.57 ± 4.32 | 50 | 24.45 ± 6.42 | <0.0001 |
| SHUNT % (%) | 215 | 35.80 ± 13.74 | 165 | 32.26 ± 10.91 | 50 | 47.49 ± 15.64 | <0.0001 |
| Hepatic Reserve (%) | 215 | 79.57 ± 14.51 | 165 | 83.78 ± 11.08 | 50 | 65.70 ± 15.89 | <0.0001 |
| Portal HFR (mL/min/kg) | 215 | 13.21 ± 6.08 | 165 | 14.64 ± 5.68 | 50 | 8.49 ± 4.88 | <0.0001 |
| Systemic HFR | 215 | 4.03 ± 0.88 | 165 | 4.20 ± 0.75 | 50 | 3.46 ± 1.04 | <0.0001 |
| (mL/min/kg) | | | | | | | |
| SHUNT V2.0 | | | | | | | |
| DSI | 215 | 18.87 ± 5.71 | 165 | 17.30 ± 4.51 | 50 | 24.05 ± 6.21 | <0.0001 |
| SHUNT % (%) | 215 | 34.10 ± 13.54 | 165 | 30.84 ± 11.20 | 50 | 44.88 ± 15.06 | <0.0001 |
| Hepatic Reserve (%) | 215 | 80.50 ± 14.41 | 165 | 84.60 ± 11.36 | 50 | 66.96 ± 15.22 | <0.0001 |
| Portal HFR (mL/min/kg) | 215 | 14.04 ± 7.22 | 165 | 15.57 ± 6.96 | 50 | 9.00 ± 5.60 | <0.0001 |
| Systemic HFR | 215 | 3.99 ± 0.87 | 165 | 4.16 ± 0.76 | 50 | 3.42 ± 0.98 | <0.0001 |
| (mL/min/kg) | | | | | | | |

^{a} Primary clinical outcome defined as liver-related death, CP progression, variceal hemorrhage, ascites, and/or encephalopathy.
^{b} Differences between subjects who did/did not experience the primary clinical outcome by t test. Bold values represent statistical significance, p < 0.05.
Abbreviations: DSI, disease severity index; HFR, hepatic filtration rate; SHUNT %, portal-systemic shunting.

Table 8 shows Year 2 cholate SHUNT test results for all subjects and by subjects who either did or did not experience the primary clinical outcome within the study period.

TABLE 8
Year 2 cholate SHUNT test results for all subjects and by subjects
who did or did not experience the primary clinical outcome.

| | | | No clinical | | | | |
|---|---|---|---|---|---|---|---|
| Test Version/ | All subjects | | outcome | | Clinical outcome ^{a} | | |
| Parameter | n | Mean ± SD | n | Mean ± SD | n | Mean ± SD | p value ^{b} |
| SHUNT V1.0 | | | | | | | |
| DSI | 187 | 20.99 ± 7.42 | 143 | 18.48 ± 5.28 | 44 | 29.11 ± 7.60 | <0.0001 |
| SHUNT % (%) | 187 | 42.50 ± 18.53 | 143 | 36.59 ± 13.63 | 44 | 61.69 ± 19.48 | <0.0001 |
| Hepatic Reserve (%) | 187 | 75.05 ± 18.71 | 143 | 81.47 ± 12.91 | 44 | 54.16 ± 19.51 | <0.0001 |
| Portal HFR | 187 | 12.15 ± 6.93 | 143 | 14.05 ± 6.57 | 44 | 5.99 ± 3.77 | <0.0001 |
| (mL/min/kg) | | | | | | | |
| Systemic HFR | 187 | 4.14 ± 1.37 | 143 | 4.46 ± 1.27 | 44 | 3.12 ± 1.19 | <0.0001 |
| (mL/min/kg) | | | | | | | |
| SHUNT V1.1 | | | | | | | |
| DSI | 187 | 20.75 ± 7.51 | 143 | 18.18 ± 5.28 | 44 | 29.09 ± 7.63 | <0.0001 |
| SHUNT % (%) | 187 | 40.88 ± 17.58 | 143 | 35.17 ± 12.68 | 44 | 59.45 ± 18.56 | <0.0001 |
| Hepatic Reserve (%) | 187 | 75.03 ± 18.92 | 143 | 81.58 ± 13.06 | 44 | 53.73 ± 19.45 | <0.0001 |
| Portal HFR | 187 | 12.19 ± 6.98 | 143 | 14.10 ± 6.63 | 44 | 6.00 ± 3.80 | <0.0001 |
| (mL/min/kg) | | | | | | | |
| Systemic HFR | 187 | 3.97 ± 1.10 | 143 | 4.27 ± 0.93 | 44 | 2.99 ± 1.04 | <0.0001 |
| (mL/min/kg) | | | | | | | |
| SHUNT V2.0 | | | | | | | |
| DSI | 187 | 20.60 ± 7.55 | 143 | 18.04 ± 5.47 | 44 | 28.94 ± 7.43 | <0.0001 |
| SHUNT % (%) | 187 | 40.07 ± 18.31 | 143 | 34.23 ± 12.91 | 44 | 59.06 ± 20.43 | <0.0001 |
| Hepatic Reserve (%) | 187 | 75.54 ± 19.01 | 143 | 82.08 ± 13.45 | 44 | 54.31 ± 19.05 | <0.0001 |
| Portal HFR | 187 | 12.60 ± 7.39 | 143 | 14.61 ± 7.07 | 44 | 6.09 ± 3.77 | <0.0001 |
| (mL/min/kg) | | | | | | | |
| Systemic HFR | 187 | 3.94 ± 1.09 | 143 | 4.24 ± 0.94 | 44 | 2.98 ± 0.99 | <0.0001 |
| (mL/min/kg) | | | | | | | |

^{a} Primary clinical outcome defined as liver-related death, CP progression, variceal hemorrhage, ascites, and/or encephalopathy.
^{b} Differences between subjects who did/did not experience the primary clinical outcome by t test.
Note:
Actual time intervals were calculated from the dates of testing. Results include subjects who experienced clinical outcome between baseline and Year 2. Bold values represent statistical significance, p < 0.05.
Abbreviations: DSI, disease severity index; HFR, hepatic filtration rate; SHUNT %, portal-systemic shunting.

### Patient/Provider RISK ACE App

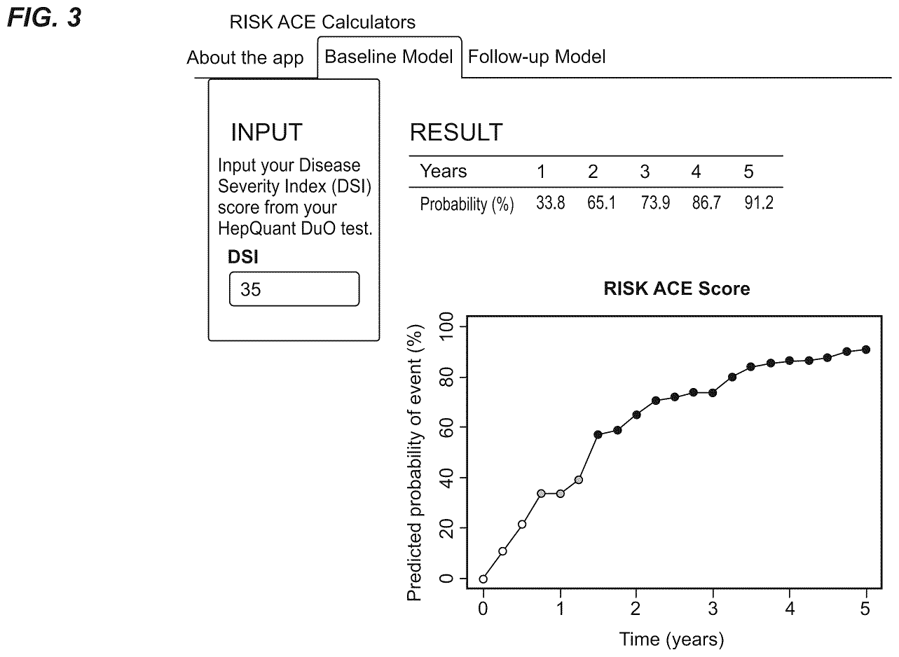

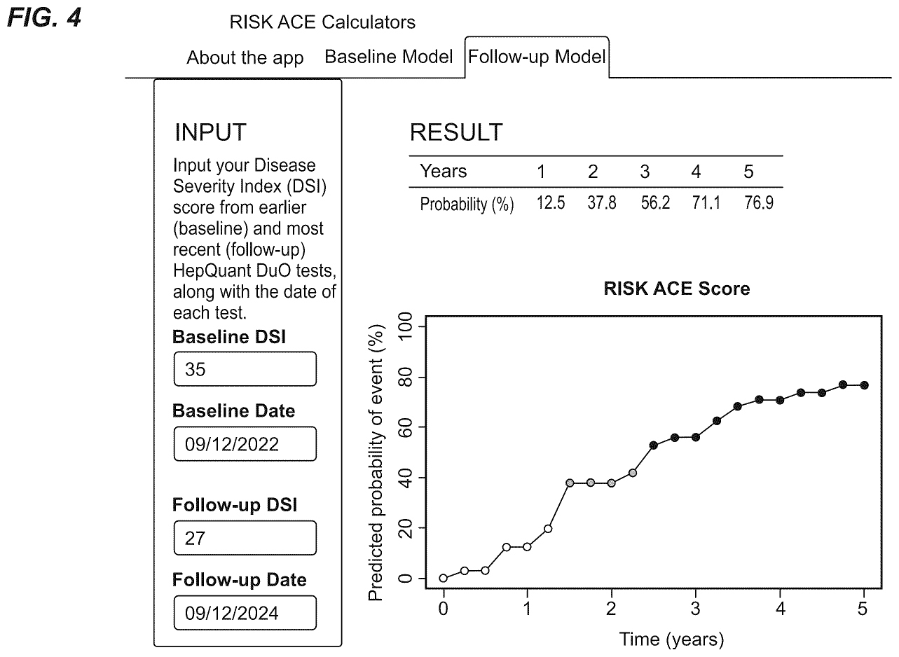

A web application (app) facilitates the use and explanation of the RISK ACE evaluation of the results of liver function testing [24]. The web app was developed using the R package, Shiny [25], and may be accessed by smart phone or computer. The RISK ACE app interface is shown in FIG. 3 and FIG. 4. FIG. 3 shows a screenshot of RISK ACE web app (Baseline, Model A). FIG. 4 shows a screenshot of RISK ACE web app (Follow-up, Model D). Patients and/or providers input DSI results, and the RISK ACE results are updated dynamically in the plots and tables.

This study demonstrated that liver function measured by the DuO oral distinguishable cholate challenge test can predict decompensation in cACLD. In addition, parameters of the test demonstrated stronger associations with clinical outcome than other laboratory tests, clinical scores, and fibrosis staging by liver biopsy (by univariate hazard ratio, Chi-square statistic). The results with DSI, SHUNT %, and Hepatic Reserve from DuO oral distinguishable cholate challenge test are consistent with the results for portal HFR and SHUNT % in prior publication of SHUNT test [19]. Everson et al., Hepatology. 2012; 55(4):1019-29.

Most models for the prediction of clinical outcome in patients with liver disease have relied on static laboratory tests (e.g., MELD), or a combination of laboratory tests and clinical assessment (CP class and score) [26-29]. Although useful in late-stage disease for prediction of short-term mortality or surgical risk, these models underperform in clinically stable, compensated patients with advanced fibrosis or cirrhosis because test values can remain in the normal range even while liver function declines to the point of decompensation.

In contrast, the DSI models utilize dynamic tests that quantify liver function and physiology. DSI indexes a patient's cholate clearance against the maximum cholate clearance of healthy controls to provide a global score of liver health [30]. Because DSI reflects the composite effect of liver cell function, liver perfusion, and portal systemic shunting it assesses effective sinusoidal perfusion and functional acinar mass [13, 31]. Since changes in function, as detected by distinguishable cholate challenge tests, occur prior to development of clinical complications or outcomes, distinguishable cholate challenge tests may be more appropriate than the existing clinical models for predicting decompensation in patients with stable cirrhosis.

One key observation from this study was that the most recent test result remains a significant predictor of clinical outcome even after adjusting for the baseline test result, as demonstrated by RISK ACE Model C which included baseline DSI and the DSI at 2-year follow-up (Table 4). Additionally, the rate of change from baseline appears to be the more predictive of risk for future clinical events (RISK ACE Model D), although additional longitudinal data may be useful to fully understand the nature of the longer-term relationship between DSI change and the risk for clinical events. These results suggest that disease progression can be better tracked by changes in liver function as quantified by DSI. Additional aspects include determination of optimal testing intervals for using RISK ACE with rate of change from baseline (Model D), and analysis with advanced modeling approaches (e.g., Cox regression model with time-dependent covariates).

The Risk for Adverse Clinical Events (RISK ACE) models provided herein accomplish a key objective: to express the results of the DuO oral distinguishable cholate challenge test in terms of the predicted risk for a clinical event as a function of time after testing. One of the challenges in interpreting the cholate challenge test results is that the relationship of DSI to risk for clinical outcome is nonlinear. The non-linearity of risk predictions means that the risk of adverse events accelerates most rapidly with increasing DSI for patients who have a higher baseline DSI. For example, the risk curve corresponding to median DSI was much lower than the Kaplan Meier curve, which was much closer to the 75^{th} percentile DSI (FIG. 1). A relatively major (10-point) increase in DSI from 4.9 to 14.8 was associated with a minor increase of 3.6% in 3-year risk (from 0.8% to 4.4%). In contrast, a much smaller increase (4-point) in DSI from 18.4 to 22.7 was associated with a 7.8% increase in 3-year risk (from 8.0% to 15.8%). These examples illustrate the importance of interpreting DSI in a patient-centric scale such as RISK ACE. This property means that the RISK ACE model appropriately expresses changes in DSI in a more familiar scale as changes in risk.

The strength of the relationships of liver function to clinical outcomes by Cox regression supports continued development of these RISK ACE models. Additional aspects includes other clinical laboratory and imaging data to optimize the estimation of clinical risk. Although the etiology of liver disease in the present analysis was chronic hepatitis C, covariates for disease progression, such as overweight, obesity, and alcohol use, were common. Additional aspects include other liver disease etiologies, including MASLD/MASH.

Of the 215 subjects, 106 (49%) had a DSI value >18.3. Fifty subjects experienced decompensation events; 43 (39%) in subjects with baseline DSI>18.3 and 7 (7%) in subjects with baseline DSI<18.3 (p<0.0001). In follow-up testing, DSI at Year 2 had closer associations with clinical outcome than DSI at baseline, with hazard ratio (HR) 1.15 (95% CI: 1.07-1.23) versus 1.09 (1.00-1.18), respectively. In multivariable Cox regression, baseline DSI (HR 1.21 [1.11-1.32]) and rate of change in DSI (HR 16.9 [3.23-88.30]), but not fibrosis stage (HR 1.20 [0.82-1.76]), were associated with clinical outcome.

Conclusions: The RISK ACE model predicted clinical outcome in patients with compensated advanced chronic liver disease (cACLD) and could estimate an individual's clinical risk. RISK ACE can assist both patients and providers in optimizing clinical management.

### Example 1. Cholate Challenge Test Predicts Clinical Benefit of Resmetirom

The objective of this study was to measure changes in liver function and physiology, as quantified by the oral cholate challenge test (HepQuant DuO), during resmetirom therapy of MASH cirrhosis. RISK ACE models were used to link changes in function to clinical risk.

MASH cirrhosis is characterized by a broad spectrum of hepatic impairment and portal-systemic shunting.

Resmetirom is an oral, once-daily, liver-directed, selective thyroid stimulating hormone receptor-beta (THR-beta) agonist that is FDA-approved for use in conjunction with diet and exercise for treatment of adults with noncirrhotic metabolic dysfunction-associated steatohepatitis (MASH), formerly known as nonalcoholic steatohepatitis (NASH), with moderate to advanced liver fibrosis (consistent with F2 to F3 fibrosis). Resmetirom is under study for use in cirrhotic MASH. Primary endpoints from MAESTRO-NAFLD-1 have been reported previously. This example reports results with the oral cholate challenge test (HepQuant DuO) test and RISK ACE models from the Phase 3 MAESTRO-NAFLD-1 trial of resmetirom in subjects with well-compensated (Child-Pugh A) MASH cirrhosis.

Methods. Thirty-two subjects enrolled in the Phase 3 open-label MAESTRO-NAFLD-1 trial with MASH cirrhosis underwent baseline dual cholate shunt testing, and 25 had subsequent retesting at 28 and 48 weeks. Blood was sampled from 0 to 90 minutes, serum concentrations measured, and parameters of function (DSI), portal-systemic shunting (SHUNT %), and clinical risk (RISK ACE) were calculated. Responders were defined by >2-point decrease in DSI, stable subjects by a ΔDSI within ±2, and non-responders by >2-point increase. MAESTRO-NAFLD-1 was registered at ClinicalTrials.gov (NCT04197479).

The MAESTRO-NAFLD-1 study was a 52-week Phase 3 trial to evaluate the safety and tolerability of resmetirom for the treatment of MASH. The study included an open-label 80 mg once daily resmetirom treatment arm in patients with well-compensated (Child-Pugh A) MASH cirrhosis.

The trial consisted of a screening period of up to 8 weeks, a 52-week treatment period, and a 4-week follow-up period. Visits were conducted every 4 weeks. Eligibility required at least three metabolic risk factors and MASH cirrhosis diagnosed on liver biopsy or according to accepted criteria.

Subjects underwent baseline testing and subsequent retesting at 28 and 48 weeks. The test was administered to fasting subjects (overnight or >5 h), and morning medications were held until completion of testing. Dual cholate shunt (HepQuant SHUNT) tests were administered, involving simultaneous dosing of 20 mg [24-¹³C](13C) cholate by IV and 40 mg [2,2,4,4-²H](d4) cholate orally and three (3) mL blood samples collected at 0, 5, 20, 45, 60, and 90 min. The results herein are reported for the oral cholate challenge test (HepQuant DuO) which utilized only the oral dose of d4-cholate, followed by blood sampling at 20 and 60 minutes. Serum concentrations of cholate isotopes were quantified using LC-MS/MS. Clinical investigators and study subjects were blinded to the results of the HepQuant cholate challenge tests, and HepQuant personnel were blinded to the subjects' clinical and laboratory information.

The methods for calculating the parameters of all HepQuant cholate challenge test versions and their within-individual reproducibility are described in detail in separate publications. (McRae et al. Clin Transl Sci. 2024; 17(4):e13786; McRae et al. Basic Clin Pharmacol Toxicol. 2024; 134(3):385-395; McRae et al. Transl Res. 2023; 252:53-63. doi:10.1016/j.trsl. 2022 Aug. 2); and McRae et al. Clin Chim Acta. 2025 Jun. 15; 574:120325.

The present example focused on the results with the DuO cholate challenge test, which involves an oral administration of 40 mg [2,2,4,4-²H](d4) cholate followed by blood samples collected at 20 and 60 minutes. Serum concentrations of cholate isotopes were quantified using LC-MS/MS, and the following test parameters were calculated. The portal cholate clearance was measured using a compartmental model, and systemic clearance was estimated using linear models and noncompartmental analysis. Specifically, in the oral-only DuO cholate challenge test version, the systemic clearance of 13C-cholate is derived rather than measured. The following test parameters were calculated: Disease Severity Index (DSI), Hepatic Reserve (%), portal hepatic filtration rates (portal HFR), systemic HFR, SHUNT %, and RISK ACE scores. The RISK ACE scores estimate the probability of adverse clinical events at or before the index time and are calculated using DSI values in Cox proportional hazards regression. In this example, two RISK ACE models were evaluated: the BASELINE model which evaluates the risk using the DSI results from a single HepQuant DuO test, and the FOLLOW-UP model which evaluates the risk at follow-up using the baseline DSI and the rate of change in DSI from baseline and is intended for monitoring applications to track liver function over time.

### Study Cohort.

Out of the 32 subjects enrolled and tested at baseline, 28 had evaluable baseline tests, 23 had evaluable W28 tests, and 23 had evaluable W48 tests.

Baseline subject characteristics, clinical scores, and laboratory values are shown in Table 12 for all subjects, and by baseline DSI≤23 (n=16) or DSI>23 (n=12).

TABLE 12
Baseline subject characteristics, clinical scores, and laboratory
values for all subjects and by baseline DSI above or below 23.

| | All Subjects | | Baseline DSI ≤23 | | Baseline DSI >23 | |
|---|---|---|---|---|---|---|
| | | Mean ± SD | | Mean ± SD | | Mean ± SD |
| | n | or n (%) | n | or n (%) | n | or n (%) |
| Characteristics | | | | | | |
| Age | 28 | 60.2 ± 8.9 | 16 | 60.4 ± 9.3 | 12 | 59.9 ± 8.7 |
| Female | 28 | 14 (50%) | 16 | 8 (50%) | 12 | 6 (50%) |
| Hispanic or Latino | 28 | 6 (21%) | 16 | 4 (25%) | 12 | 2 (17%) |
| Body Mass Index | 27 | 37.3 ± 8.5 | 16 | 34.9 ± 5.5 | 11 | 40.7 ± 11.1 |
| Weight (kg) | 28 | 107.1 ± 25.6 | 16 | 101.3 ± 21.3 | 12 | 114.9 ± 29.5 |
| Diabetes | 28 | 19 (68%) | 16 | 13 (81%) | 12 | 6 (50%) |
| Hypothyroid | 28 | 5 (18%) | 16 | 4 (25%) | 12 | 1 (8%) |
| Hypertension | 28 | 25 (89%) | 16 | 15 (94%) | 12 | 10 (83%) |
| Medications | | | | | | |
| Statin | 28 | 16 (57%) | 16 | 13 (81%) | 12 | 3 (25%) |
| GLP-1 | 28 | 6 (21%) | 16 | 4 (25%) | 12 | 2 (17%) |
| Insulin | 28 | 4 (14%) | 16 | 3 (19%) | 12 | 1 (8%) |
| SGLT2 | 28 | 4 (14%) | 16 | 4 (25%) | 12 | 0 (0%) |
| Thyroxine | 28 | 6 (21%) | 16 | 4 (25%) | 12 | 2 (17%) |
| Imaging and non- | | | | | | |
| invasive measures | | | | | | |
| FibroScan VCTE (kPa) | 25 | 30.8 ± 17.2 | 15 | 27.1 ± 17.1 | 10 | 36.3 ± 16.5 |
| CAP (dB/m) | 25 | 333.6 ± 54.9 | 15 | 325.7 ± 55.3 | 10 | 345.4 ± 55.0 |
| MRE (kPa) | 25 | 5.6 ± 1.9 | 15 | 5.4 ± 2.0 | 10 | 5.9 ± 1.7 |
| Hepatic Fat Fraction by | 26 | 9.2 ± 4.3 | 16 | 8.8 ± 3.4 | 10 | 9.8 ± 5.7 |
| Laboratory values and | | | | | | |
| clinical scores | | | | | | |
| Glucose (mg/dL) | 28 | 130.5 ± 30.7 | 16 | 126.4 ± 31.6 | 12 | 136.1 ± 29.8 |
| HOMA-IR | 28 | 14.8 ± 20.8 | 16 | 7.6 ± 2.5 | 12 | 24.5 ± 29.6 |
| Total Bilirubin (mg/dL) | 28 | 0.9 ± 0.6 | 16 | 0.7 ± 0.3 | 12 | 1.1 ± 0.7 |
| ALP (U/L) | 28 | 77.4 ± 25.1 | 16 | 72.6 ± 24.6 | 12 | 83.8 ± 25.5 |
| ALT (U/L) | 28 | 37.1 ± 16.5 | 16 | 35.3 ± 17.8 | 12 | 39.6 ± 15.2 |
| AST (U/L) | 28 | 36.3 ± 15.8 | 16 | 31.4 ± 15.7 | 12 | 42.8 ± 14.0 |
| GGT (U/L) | 28 | 119.6 ± 115.1 | 16 | 85.4 ± 78.3 | 12 | 165.3 ± 142.1 |
| Platelets (10³/μL) | 27 | 145.9 ± 77.1 | 15 | 175.8 ± 87.6 | 12 | 108.6 ± 39.5 |
| Albumin (g/dL) | 28 | 4.1 ± 0.3 | 16 | 4.2 ± 0.3 | 12 | 3.9 ± 0.4 |
| PRO-C3 (ng/mL) | 27 | 53.6 ± 24.2 | 16 | 48.4 ± 22.1 | 11 | 61.2 ± 26.1 |
| FIB-4 | 27 | 3.2 ± 2.1 | 15 | 2.3 ± 1.3 | 12 | 4.4 ± 2.2 |
| ELF Score | 28 | 10.5 ± 1.0 | 16 | 10.2 ± 0.9 | 12 | 10.8 ± 1.1 |
| MELD Score | 27 | 8.4 ± 1.9 | 15 | 7.9 ± 1.2 | 12 | 9.1 ± 2.4 |

Abbreviations: ALP, alkaline phosphatase; ALT, alanine transaminase; AST, aspartate transaminase; CAP, controlled attenuation parameter; DSI, disease severity index; ELF Score, Enhanced Liver Fibrosis score; FIB-4, Fibrosis-4 score; GGT, gamma-glutamyl transferase; GLP-1, glucagon-like peptide-1 agonists; MELD Score, Model for End-Stage Liver Disease; MRE, magnetic resonance elastography; SGLT2, sodium-glucose cotransporter 2 inhibitors; VCTE, vibration controlled transient elastography.

The cohort had an average MELD score of 8.4, consistent with well-compensated CP A cirrhosis. The average age was 60±±9 years, weight 107±26 kg, and BMJ 37±9 kg m⁻² Fifty percent were female, and 2100 Hispanic or Latino. Sixty-eight percent had diabetes. Subjects with better liver function at baseline (DSI≤23) were more likely to be taking statins (81%) versus those with DSI>23 (25%). Subjects with baseline DSI>23, compared to subjects with baseline DSI≤23, had higher kPa by FibroScan (but not by MRE), similar levels of liver fat, and worse results for bilirubin, albumin, platelet count, FIB-4, and MELD score (but not ELF score).

Subject characteristics were compared between those with baseline DSI≤23 versus >23. In the SHUNT-V pivotal study, investigators validated the cutoffs of SHUNT %≤30% and DSI≤18.3 for ruling out large esophageal varices; (Hassanein et al., Aliment Pharmacol Ther. 2024; 60(2):246-56; Shiffman et al., Aliment Pharmacol Ther. 2025 January; 61 (1):75-87) DSI≤18.3 indicated low risk (<4.7%), DSI 18.3 to ≤23 moderate risk (<9.4%), and DSI values >23 high risk for varices. For reference, the normal range of DSI was <11.6, defined by results from healthy controls with lean body mass (<25 kg/m², n=26). (Helmke et al., J Appl Lab Med. 2024; 9(6):1028-1039; Helmke et al., Gastro Hep Advances. 2024; 3(7):944-953).

The two RISK ACE models (BASELINE and FOLLOW-UP) used in this example were developed from serial studies of 215 subjects with compensated advanced chronic HCV followed for up to 7.9 years. (Kittelson et al., Eur J Intern Med. 2024; 132:160-163. doi:10.1016/j.ejim.2024.11.029). The clinical outcomes for these models included liver-related death, sustained 2-point increase in Child-Pugh score, variceal hemorrhage, ascites, and encephalopathy. In the BASELINE model, risk for clinical event was directly related to baseline DSI. In the FOLLOW-UP model, risk increases with DSI but decreases if DSI is stable or decreased.

For analysis of the MAESTRO-NAFLD-1 results, RISK ACE scores were calculated using the BASELINE model for baseline DSI and the FOLLOW-UP model for W48 DSI. The change in RISK ACE between follow-up and baseline were evaluated for all subjects and by tertiles of baseline DSI (low DSI≤18.3, moderate DSI 18.3-23, high DSI>23) with p values calculated from the bootstrapped (n=5000) distribution of the differences in estimated risk for clinical events between W48 and baseline representing the proportion of bootstrapped differences ≥0.

### Results

Responder analysis was completed for baseline to Week 28 (W28) and baseline to Week 48 (W48). Subjects responding to treatment and having a more than 2-point decrease in DSI were defined as responders, stable subjects had ±2 DSI difference, and subjects having more than a 2-point increase in DSI were defined as non-responders. The threshold of 2 DSI units was based on previous individual reproducibility studies (McRae et al. Clin Transl Sci. 2024; 17(4):e13786; Burton et al. Transl Res. 2021; 233:5-15) of 94 subjects encompassing healthy persons and subjects with MASH, HCV, and primary sclerosing cholangitis, where the minimum detectable difference in DSI was 1.7. Proportions of responders between W28 and W48 were compared using McNemar's test because the responder proportions are repeated measurements on the same individuals. Table 13 shows cross-tabulation of 28- and 48-week response, as defined by a change of 2 DSI units, among the 21 participants with baseline, week 28, and week 48 results.

Table 13. Cross-tabulation of 28- and 48-week response, as defined by a change of 2 DSI units, among the 21 participants with baseline, week 28, and week 48 results.

| Week 28 | Week 48 Response | | | | |
|---|---|---|---|---|---|
| Response | Non-responder | Stable | Responder | NA ^{a} | TOTAL |
| Non-responder | 0 | 2 | 1 | 0 | 3 |
| Stable | 3 | 8 | 4 | 2 | 15 |
| Responder | 0 | 0 | 3 | 0 | 3 |
| NA ^{a} | 1 | 0 | 1 | — | — |
| TOTAL | 3 | 10 | 8 | — | 21 |

^{a} NA, data not collected for one of either Week 28 or Week 48 visits

The difference from baseline for DSI and the estimated 48-week RISK ACE at W48 in all subjects and subjects stratified by baseline risk status (DSI) is shown in Table 14.

TABLE 14
Difference from baseline for DSI and the estimated 48-week RISK ACE at W
48 in all subjects and subjects stratified by baseline risk status (DSI)

| | | | | | Change in 48-week RISK | | |
|---|---|---|---|---|---|---|---|
| | | Change in DSI at W 48 | | | ACE (%) at W 48 | | |
| Baseline risk category | n pairs | Mean (SD) | | p value ^{a} | Mean (SD) | | p value ^{b} |
| All subjects | 23 | −0.93 | (4.75) | 0.3598 | −3.98 | (11.88) | 0.0406 |
| Low risk (DSI ≤ 18.3) | 7 | −0.02 | (5.43) | 0.9936 | −0.14 | (3.28) | 0.3718 |
| Moderate risk (DSI 18.3 to | 5 | 0.13 | (1.96) | 0.8912 | −1.66 | (0.54) | <0.0001 |
| 23) | | | | | | | |
| High risk (DSI > 23) | 11 | −1.98 | (5.29) | 0.2421 | −7.48 | (16.66) | 0.0542 |

^{a} Calculated from paired t test
^{b} Calculated from the bootstrapped (n = 5000) distribution of the differences in estimated risk for clinical events between W 48 from baseline representing the proportion of bootstrapped differences ≥ 0
Abbreviations: DSI, disease severity index; RISK ACE, risk of adverse clinical events; W 48, Week 48.

The significant reduction in the estimated clinical risk by RISK ACE was paralleled by non-significant trends in reduction in mean DSI. Other clinical models, MELD and Child-Pugh score, have shown that in comparison to worsening, stability of these scores has been associated with reduced risk for clinical outcome. In this study, this phenomenon was captured by the RISK ACE score despite the overall lack of statistically significant reductions in mean DSI. After 48 weeks of resmetirom, 83% of subjects either showed improvement or stability in their DSI. This is consistent with the mechanism of action of resmetirom, a liver directed thyroid hormone receptor-β agonist, whose immediate effect would reduce intrahepatic triglycerides and inflammation, but in the long-term remodel fibrosis, further reducing DSI.

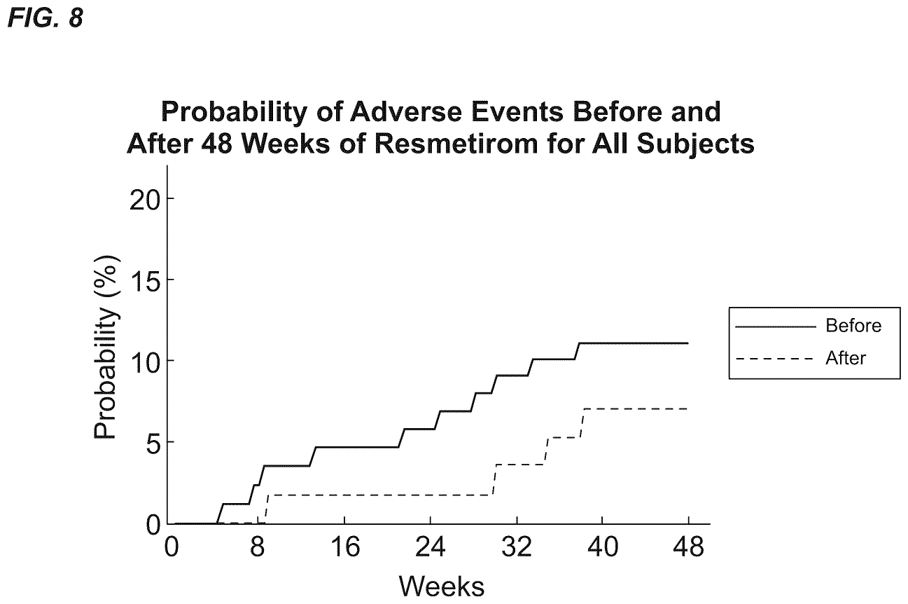

The probability of adverse clinical events by RISK ACE before and after 48 weeks of resmetirom for all subjects (n=23) is shown in FIG. 8. The solid line represents the baseline DSI and probability (%) of clinical outcome prior to treatment with resmetirom. Dashed line represents the effect after 48 weeks treatment with resmetirom on probability (%) of clinical outcome risk based on week 48 DSI measurement. RISK ACE decreased after week 48 of treatment in 19 of 23 subjects, with a significant reduction in absolute risk of −4.0%, p=0.041.

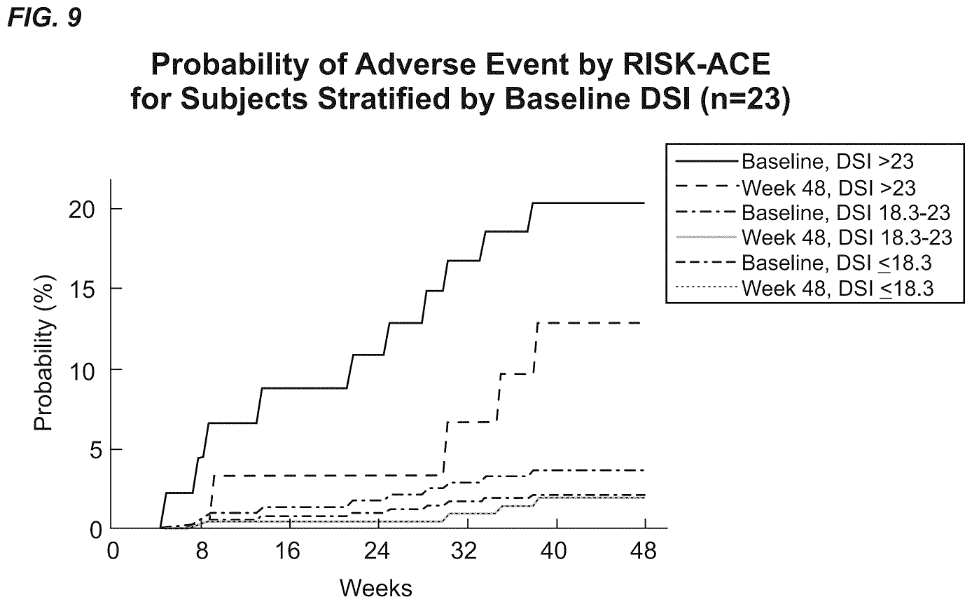

The probability of adverse clinical events by RISK ACE for all subjects with follow-up at 48 weeks and stratified by baseline DSI (n=23), with DSI>23 indicating high risk, DSI 18.3-23 indicating moderate risk, and DSI<18.3 indicating low risk is shown in FIG. 9.

Baseline DSI (22±7) and SHUNT % (36%±16%) were consistent with significant functional impairment and portal-systemic shunting. Responder rates were 14% by 28 weeks and 38% by 48 weeks (p=0.0254). After 28 weeks, 87% of subjects were either stable or showed improvement. After 48 weeks, 83% of subjects were either stable or showed improvement, with a significant reduction in mean RISK ACE from baseline (−4.0%, p=0.041). Subjects with the greatest hepatic impairment (n=12, baseline DSI≥23) showed the greatest reduction in RISK ACE (−7.5%, p=0.053).

In an open-label study of treatment of MASH cirrhosis with Resmetirom (n=23), RISK ACE predicted an event rate of 7% which was the event rate observed in the greater MAESTRO NAFLD-1-OLE F4 cohort (n=122). In summary of the 113 patients the clinical outcomes included 2 patients who died due to COVID and metastatic cancer, and 6 patients who experienced decompensation events through period of follow-up leading to an adverse clinical outcome of about 7%.

### Conclusions

This study demonstrated that 48 weeks of resmetirom treatment of subjects with MASH cirrhosis was associated with reduction in the estimated risk for clinical outcome as measured by RISK ACE.

In conclusion, the risk assessment tools, DSI and RISK ACE from the DuO test, was used to estimate that 48 weeks of resmetirom in CP A MASH cirrhosis is projected to reduce rates of clinical outcomes in this patient population. DSI, SHUNT %, and RISK ACE may provide early estimates of potential clinical benefit with resmetirom treatment.

### Example 2. Rencofilstat Treatment Improves Liver Function in MASH With Advanced Fibrosis as Quantified by DuO Cholate Challenge Test

In this example, seventy subjects with suspected ≥F3 MASH, defined from liver biopsy or AGILE 3+≥0.53, were randomized to rencofilstat 75 mg/d (n=24), 150 mg/d (n=23) or 225 mg/d (n=23), and tested by HepQuant at baseline, 60 and 120 days. The DuO version included oral dosing of d4-cholate and two blood samples (20 and 60 min). The DuO cholate challenge test disease severity index (DSI) and portal-systemic shunting fraction (SHUNT %) were evaluated for changes from baseline at 60 and 120 days of rencofilstat treatment. Details of the study are available in Harrison et al., Liver International, 2025; 45:e70036.

Rencofilstat inhibits cyclophilin to reduce hepatic inflammation and fibrosis, which, in turn, could improve liver function and reduce portal-systemic shunting. Since the SHUNT % and cholate DSI measured by DuO cholate challenge test quantifies liver function and portal-systemic shunting, it was used to measure the hepatic effects of rencofilstat treatment of MASH with advanced fibrosis.

Results: Across all subjects, there was a significant decrease in SHUNT % both at Day 60 (−1.67%, p=0.0156) and Day 120 (−1.55%, p=0.0441). In the 225 mg rencofilstat arm, 56% of subjects (10/18) were responders by Day 120 (p=0.0549), and their DSIs improved with a mean change of −1.61 (p=0.0190). Across all treatment arms, subjects with DSI>18.3 at baseline had the greatest improvement with treatment (ΔDSI=−2.59, p=0.0053).

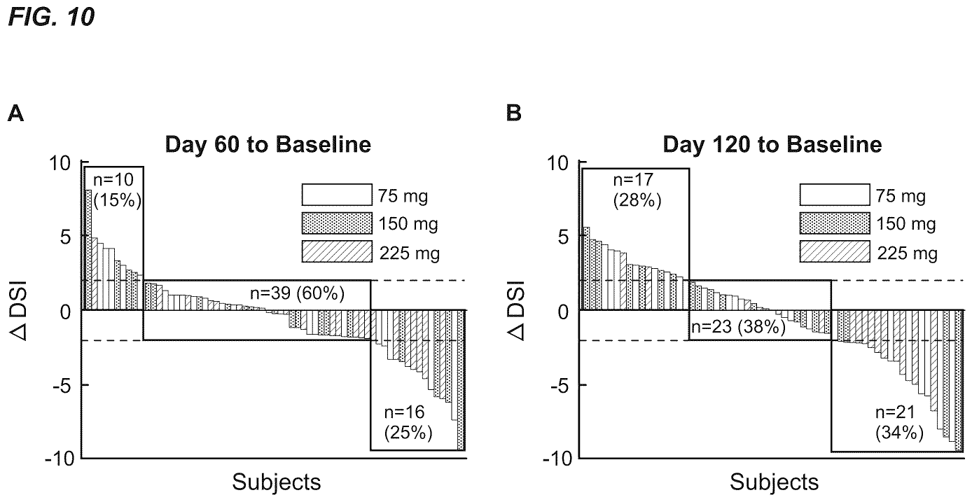

FIG. 10 shows two waterfall plots of change in cholate disease severity index (DSI) from baseline in subjects taking 75 mg, 150 mg, or 225 mg Rencofilstat daily measured by DuO cholate challenge test at Day 60 (Panel A) and Day 120 (Panel B). The horizontal lines represent limits of ±2 DSI units for responder or worsening thresholds.

Conclusion: The decreases in DSI as well as SHUNT % suggest that rencofilstat 225 mg/d improves hepatic function and portal-systemic shunting. The HepQuant DuO cholate challenge test is easy to administer, well-tolerated and a useful tool for detecting the hepatic effects of treatment.

### CLAUSES

Clause 1. A method of determining risk for an adverse clinical outcome in a patient having a chronic liver disease, the method comprising
obtaining a baseline test value from a distinguishable cholate challenge test in the patient;
entering the patient baseline test value to a data base comprising comparative distinguishable cholate challenge test values computed from measurements of labeled cholate concentrations in blood samples obtained over time from a known population of chronic liver disease subjects that had been followed prospectively for clinical events; and
computing the individual risk for clinical outcome in the patient using the baseline test value as a predictor, optionally wherein the risk for clinical outcome is displayed over a period of time of at least 2 years, at least 3 years, or at least 4 years.

Clause 2. The method of clause 1, wherein the data base comprising comparative test values was prepared from a Cox proportional hazards regression model, or similar statistical methods including Cox proportional hazards regression models with time-varying covariates, parametric survival models, or Kaplan Meier, to predict risk for adverse clinical events (RISK ACE) using the comparative distinguishable cholate challenge test values from the distinguishable cholate challenge test.

Clause 3. The method of clause 1 or 2, wherein the distinguishable cholate challenge test is selected from the group consisting of a liver disease severity index (DSI) test, cholate SHUNT test, an oral only distinguishable cholate challenge test (DuO), Hepatic Reserve test, portal hepatic filtration rate (HFR_{P}), and systemic hepatic filtration rate (HFRs), optionally wherein the distinguishable cholate challenge test comprises a DSI test or a cholate SHUNT test.

Clause 4. The method of any one of clauses 1-3, wherein the patient is suffering from a compensated advanced chronic liver disease (cACLD).

Clause 5. The method of any one of clauses 1-4, wherein the clinical outcome is selected from the group consisting of liver-related death, hepatocellular carcinoma (HCC), Child-Pugh progression, variceal hemorrhage, ascites, and hepatic encephalopathy, and optionally, other manifestations of hepatic decompensation selected from the group consisting of jaundice, coagulopathy, nutritional deficiencies, muscle wasting, sepsis, and spontaneous bacterial peritonitis.

Clause 6. The method of any one of clauses 1-5, wherein the comparative test values were collected in at least two different time points from the population of known chronic liver disease subjects.

Clause 7. The method of any one of clauses 1-6, further comprising
obtaining a follow-up distinguishable cholate challenge test value in the patient after a first period of time and entering the follow-up test value to the data base comprising the comparative test values;
entering date the baseline test value was obtained and date the follow-up test value was obtained; and
computing the individual risk for clinical outcome in the patient using the baseline test value, and subsequent change from baseline in the follow-up test value as predictors.

Clause 8. The method of any one of clauses 1-6, further comprising
obtaining a follow-up distinguishable cholate challenge test value in the patient and entering the follow-up test value to the data base comprising the comparative test values;
entering date the baseline test value was obtained and date the follow-up test value was obtained; and
computing the individual risk for clinical outcome in the patient using the baseline test value, and the follow-up test value as predictors.

Clause 9. The method of any one of clauses 1-6, further comprising
obtaining a follow-up distinguishable cholate challenge test value in the patient and entering the follow-up test value to the data base comprising the comparative test values;
entering the dates the baseline and follow-up test values were obtained in the patient; and
computing the individual risk for clinical outcome in the patient using the baseline test value and change between the baseline and follow-up test values divided by the time in months between the baseline test and follow-up test to obtain rate of change between baseline and follow-up test values as predictors.

Clause 10. The method of any one of clauses 1-9, wherein the distinguishable cholate compound is an isotope labeled distinguishable cholate compound.

Clause 11. The method of any one of clauses 1-10, wherein the distinguishable cholate compound is a stable isotope labeled distinguishable cholate compound.

Clause 12. The method of clause 11, wherein the stable isotope labeled cholate compound is selected from the group consisting of d4-cholate, d5-cholate, d2 cholate, and ¹³C-cholate, optionally wherein the d4-cholate is 2,2,4,4-d4 cholate, optionally wherein the d5-cholate is 2,2,3,4,4-cholate, and further optionally wherein the 13C cholate is 24-¹³C-cholate.

Clause 13. The method of any one of clauses 1-12, wherein the chronic liver disease is selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

Clause 14. The method of any one of clauses 1-13, wherein the patient or subject is a human patient or subject.

Clause 15. The method of any one of clauses 1-14, comprising
obtaining blood or serum sample concentration data of an orally administered distinguishable cholate compound collected from the patient at two time points after oral administration;
measuring the area under the curve of the blood or serum concentrations of the orally administered distinguishable cholate compound (AUCoral) in the patient comprising
simulating a full oral clearance curve using a compartmental model of oral cholate clearance, the compartmental model comprising body mass index (BMI), body weight (BW), and optionally hematocrit (Hct) input values in the patient;
calculating the area comprising trapezoidal numerical integration to obtain the AUCoral; and
calculating one or more distinguishable cholate challenge test results in the patient using the AUCoral, wherein the test results are associated with liver function in the patient.

Clause 16. The method of clause 15, wherein the obtaining concentration data of the administered distinguishable cholate compound at the two time points comprises
receiving first and second blood or serum samples that had been collected from the patient at first and second time points following a single oral dose of a first distinguishable cholate compound; and
analyzing the samples to obtain the oral concentration data at the first and second time points, optionally wherein the blood or serum samples had been collected within about 180 minutes, 120 minutes, 90 minutes, or within about 75 minutes, after the oral administration.

Clause 17. The method of clause 16, wherein the first and second blood or serum samples had been collected from the subject between at least about 5 min to about 75 min, 10 min to 70 min, 20 min to 60 min, 25 min to 55 min, 30 min to 50 min, 35 to 45 min, or about 40 min apart.

Clause 18. The method of clause 16 or 17, wherein the first and second blood or serum samples had been collected from the patient at about 20 min and about 60 min following the oral administration, respectively.

Clause 19. The method of any one of clauses 15-18, further comprising estimating an area under the curve of blood or serum concentrations of an intravenously administered distinguishable cholate compound (AUCiv); and calculating a DSI value or SHUNT test value in the patent using the AUCoral and estimated AUCiv values.

Clause 20. The method of clause 19, wherein the estimating the AUCiv comprises a linear regression model, optionally wherein the linear regression model comprises equation 11A:

$${AUC}_{IV} = {β}_{0} + {β}_{BW} \cdot BW + {β}_{PO , 20} \cdot {C}_{PO , 20} + {β}_{PO , 60} \cdot {C}_{PO , 60} + {β}_{HFR , P} \cdot {HFR}_{P} , \qquad Eqn . 11 A ,$$

wherein
β₀ is an intercept coefficient, optionally wherein the intercept coefficient is 161.972;
B_{BW} is a body weight coefficient, optionally wherein the body weight coefficient is 0.6459;
β_{PO,20} is an orally administered distinguishable cholate concentration coefficient at a first time point, optionally wherein the β_{PO,20} is 16.9249;
C_{PO,20} is the orally administered distinguishable cholate concentration at the first time point;
β_{PO,60} is an orally administered distinguishable cholate concentration coefficient at a second time point, optionally wherein the β_{PO,60} is 89.2405;
C_{PO,60} is an orally administered distinguishable cholate concentration at the second time point; and
β_{HFR, P} is a portal HFR coefficient, optionally wherein the portal HFR coefficient is −0.4755.

Clause 21. The method of clause 19 or 20, wherein the estimating the AUCiv comprises
exponential fitting the intravenous concentration data to a systemic cholate clearance curve comprising fast, moderate, and slow phases of clearance over at least about 180 min after the iv administration of the intravenous dose.

Clause 22. The method of clause 21, wherein the fitting to systemic cholate clearance curve fast phase (Y₀) is calculated according to equation 20:

$${Y}_{0} = {C}_{0} \cdot {e}^{- {k}_{fast} \cdot t} , \qquad Eqn . 20$$

wherein
t=time (0 to 20 min);
C₀ is the initial concentration of intravenously administered distinguishable cholate compound,
C₂₀ is the measured 20-minute concentration of intravenously administered distinguishable cholate compound; and
k_{fast} is the rate of elimination in the fast phase estimated by equation 18:

$${k}_{fast} = - \frac{ln ( {C}_{20} ) - ln ( {C}_{0} )}{{T}_{20}} , \qquad Eqn . 18$$

optionally wherein
T₂₀ is the actual time recorded for the 20-minute sample;
C₀ is estimated according to equation 17;

$${C}_{0} = \frac{{D}_{IV}}{{V}_{d} \cdot BW} , \qquad Eqn . 17$$

D_{IV} is the intravenous dose of third distinguishable cholate;
BW is subject body weight (kg); and
Vd is the volume of distribution (V_{d}) in L per kg body weight, calculated according to equation 16A:

$$TPV = {V}_{d} = \frac{0.07}{\sqrt{BMI / 22}} \cdot ( 1 - Hct ) , \qquad Eqn . 16 A$$

wherein TPV is total plasma volume, BMI is body mass index, and Hct is hematocrit in the subject.

Clause 23. The method of clause 21 or 22, wherein the fitting to systemic cholate clearance curve moderate phase (Y₁) is calculated according to equation 21:

$${Y}_{1} = \frac{{C}_{20}}{{e}^{- {k}_{mod} \cdot 20}} \cdot {e}^{- {k}_{mod} \cdot t} , \qquad Eqn . 21$$

t=time (20-60 min);
k_{mod} is the rate of elimination in the moderate phase estimated by equation 19:

$${k}_{mod} = - \frac{ln ( {C}_{60} ) - ln ( {C}_{20} )}{{T}_{60} - {T}_{20}} , \qquad Eqn . 19$$

wherein
T₂₀ is the actual time recorded for the 20-minute sample;
T₆₀ is the actual time recorded for the 60-minute sample; C₂₀ is the concentration at 20 minutes;
C₆₀ is the concentration at 60 minutes.

Clause 24. The method of any one of clauses 21-23, wherein the fitting to systemic cholate clearance curve slow phase (Y₂) is calculated according to equation 22:

$${Y}_{2} = \frac{{C}_{60}}{{e}^{- {k}_{slow} \cdot {T}_{60}}} \cdot {e}^{- {k}_{slow} \cdot t} , \qquad Eqn . 22$$

wherein
t=time (60-180 min);
C₆₀ is the 60-minute concentration of intravenously administered distinguishable cholate; and
k_{slow} is the rate of elimination in the slow phase estimated by a mean value from a multiplicity of CLD patients, optionally wherein k_{slow} is 0.018 min⁻¹.

Clause 25. The method according to any one of clauses 21 to 24, wherein the areas under each of the three exponential curve fits are calculated by trapezoidal numerical integration and summed to estimate the AUC_{IV}.

Clause 26. A computer program product, comprising a non-transitory and tangible computer-readable storage medium whose contents include a program with instructions being executed on a processor so as to perform a method for determining risk for an adverse clinical outcome in a patient having a chronic liver disease, the method comprising
obtaining a baseline test value from a distinguishable cholate challenge test in the patient;
entering the baseline patient test value to a data base comprising comparative distinguishable cholate challenge test values computed from measurements of labeled cholate concentrations in blood samples obtained over time from a known population of chronic liver disease subjects that had been followed prospectively for clinical events; and
computing the individual risk for clinical outcome in the patient using the baseline test value as a predictor, optionally wherein the risk for clinical outcome is displayed over a period of time of at least 2 years, at least 3 years, or at least 4 years.

Clause 27. A method of analyzing a response to a treatment of a chronic liver disease in a patient in need thereof, comprising
determining a baseline cholate Disease Severity Index (DSI) value in the patient having a chronic liver disease prior to the treatment;
administering the treatment to the patient for a period of time; and
determining a follow-up cholate Disease Severity Index (DSI) value in the patient after the period of treatment time, wherein a treatment responder exhibits >2-point decrease in the follow-up DSI value, a stable subject exhibits a ΔDSI within ±2-points, and a non-responder exhibits >2-point increase compared to the baseline DSI value.

Clause 28. The method of clause 27, wherein the period of time is from 2 weeks to 104 weeks, 4 weeks to 78 weeks, 8 weeks to 52 weeks, or 28 weeks to 48 weeks.

Clause 29. The method of clause 27 or 28, wherein the determining the baseline cholate DSI value in the patient comprises
obtaining blood or serum sample concentration data of an orally administered distinguishable cholate compound collected from the patient at two time points after oral administration;
measuring the area under the curve of the blood or serum concentrations of the orally administered distinguishable cholate compound (AUCoral) in the patient comprising
simulating a full oral clearance curve using a compartmental model of oral cholate clearance, the compartmental model comprising body mass index (BMI), body weight (BW), and optionally hematocrit (Hct) input values in the patient, and
calculating the area comprising trapezoidal numerical integration to obtain the AUCoral;
estimating an area under the curve of blood or serum concentrations of an intravenously administered distinguishable cholate compound (AUCiv); and
calculating the DSI value in the patent using the AUCoral and estimated AUCiv values.

Clause 30. The method of clause 29, wherein the obtaining concentration data of the orally administered distinguishable cholate compound at the two time points comprises
receiving first and second blood or serum samples that had been collected from the patient at first and second time points following a single oral dose of a first distinguishable cholate compound; and
analyzing the samples to obtain the oral concentration data at the first and second time points, optionally
wherein the blood or serum samples had been collected within about 180 minutes, 120 minutes, 90 minutes, within about 75 minutes, or within about 60 minutes after the oral administration.

Clause 31. The method of clause 30, wherein the first and second blood or serum samples had been collected from the subject between at least about 5 min to about 90 min, 10 min to 75 min, 20 min to 60 min, 25 min to 55 min, 30 min to 50 min, 35 to 45 min, or about 40 min apart.

Clause 32. The method of clause 30 or 31, wherein the first and second blood or serum samples had been collected from the subject at about 20 min and about 60 min following the oral administration, respectively.

The above specification, examples and data provide a complete description of the methods of the invention. Since many embodiments of the invention can be made without departing from the spirit and scope of the invention, the invention resides in the claims hereinafter appended.

### REFERENCES

ADDIN EN.REFLIST 1. Engelmann C, Clària J, Szabo G, Bosch J, Bernardi M. Pathophysiology of decompensated cirrhosis: Portal hypertension, circulatory dysfunction, inflammation, metabolism and mitochondrial dysfunction. J Hepatol. 2021; 75:S49-S66. doi: 10.1016/j.jhep.2021.01.002.
2. D'Amico G, Morabito A, D'Amico M, Pasta L, Malizia G, Rebora P, et al. Clinical states of cirrhosis and competing risks. J Hepatol. 2018; 68(3):563-76. doi: 10.1016/j.jhep.2017.10.020.
3. Zipprich A, Garcia-Tsao G, Rogowski S, Fleig W E, Seufferlein T, Dollinger M M. Prognostic indicators of survival in patients with compensated and decompensated cirrhosis. Liver Int. 2012; 32(9):1407-14. doi: 10.1111/j.1478-3231.2012.02830.x.
4. Abraldes J G, Garcia-Tsao G. Simple Clinical Tools to Predict Decompensation in Patients With Compensated Cirrhosis: An Unmet Need. Clin Gastroenterol Hepatol. 2019; 17(11):2179-81. doi: 10.1016/j.cgh.2019.04.026.
5. Guha I N, Harris R, Berhane S, Dillon A, Coffey L, James M W, et al. Validation of a Model for Identification of Patients With Compensated Cirrhosis at High Risk of Decompensation. Clin Gastroenterol Hepatol. 2019; 17(11):2330-8.el. doi: 10.1016/j.cgh.2019.01.042.
6. Dulai P S, Singh S, Patel J, Soni M, Prokop L J, Younossi Z, et al. Increased risk of mortality by fibrosis stage in nonalcoholic fatty liver disease: Systematic review and meta-analysis. Hepatology. 2017; 65(5).
7. Yano M, Kumada H, Kage M, Ikeda K, Shimamatsu K, Inoue O, et al. The long-term pathological evolution of chronic hepatitis C. Hepatology. 1996; 23(6):1334-40. doi: 10.1002/hep.510230607.
8. Ripoll C, Groszmann R, Garcia-Tsao G, Grace N, Burroughs A, Planas R, et al. Hepatic Venous Pressure Gradient Predicts Clinical Decompensation in Patients With Compensated Cirrhosis. Gastroenterology. 2007; 133(2):481-8. doi: 10.1053/j.gastro.2007.05.024.
9. Garcia-Tsao G, Groszmann R J, Fisher R L, Conn H O, Atterbury C E, Glickman M. Portal pressure, presence of gastroesophageal varices and variceal bleeding. Hepatology. 1985; 5(3):419-24. doi: 10.1002/hep.1840050313.
10. Blasco A, Forns X, Carrión J A, García-Pagán J C, Gilabert R, Rimola A, et al. Hepatic venous pressure gradient identifies patients at risk of severe hepatitis C recurrence after liver transplantation. Hepatology. 2006; 43(3):492-9. doi: 10.1002/hep.21090.
11. de Franchis R, Bosch J, Garcia-Tsao G, Reiberger T, Ripoll C, Abraldes J G, et al. Baveno VII—Renewing consensus in portal hypertension. J Hepatol. 2022; 76(4):959-74. doi: 10.1016/j.jhep.2021.12.022.
12. Everson G T, Martucci M A, Shiffman M L, Sterling R K, Morgan T R, Hoefs J C, et al. Portal-systemic shunting in patients with fibrosis or cirrhosis due to chronic hepatitis C: the minimal model for measuring cholate clearances and shunt. Aliment Pharmacol Ther. 2007; 26(3):401-10. doi: 10.1111/j.1365-2036.2007.03389.x.
13. McRae M P, Kittelson J, Helmke S M, Everson G T. Within individual reproducibility of a dual sample oral cholate challenge test (DuO) and other simplified versions of the HepQuant test. Clin Transl Sci. 2024; 17(4):e13786. doi: 10.1111/cts.13786.
14. McRae M P, Kittelson J, Helmke S M, Everson G T. Advances in noninvasive measurement of liver function and physiology: The HepQuant DuO test. Basic Clin Pharmacol Toxicol. 2024; 134(3):385-95. doi: 10.1111/bcpt.13980.
15. Hassanein T, Keaveny A P, Mantry P, Smith A D, McRae M P, Kittelson J, et al. Liver function and portal-systemic shunting quantified by the oral cholate challenge test and risk for large oesophageal varices. Aliment Pharmacol Ther. 2024; 60(2):246-56. doi: 10.1111/apt.18054.
16. Everson G T, Shiffman M L, Morgan T R, Hoefs J C, Sterling R K, Wagner D A, et al. The spectrum of hepatic functional impairment in compensated chronic hepatitis C: results from the Hepatitis C Anti-viral Long-term Treatment against Cirrhosis Trial. Aliment Pharmacol Ther. 2008; 27(9):798-809. doi: 10.1111/j.1365-2036.2008.03639.x.
17. Everson G T, Shiffman M L, Hoefs J C, Morgan T R, Sterling R K, Wagner D A, et al. Quantitative tests of liver function measure hepatic improvement after sustained virological response: results from the HALT-C trial. Aliment Pharmacol Ther. 2009; 29(5):589-601. doi: 10.1111/j.1365-2036.2008.03908.x.
18. Fallahzadeh M A, Hansen D J, Trotter J F, Everson G T, Saracino G, Rahimi R S, et al. Predicting clinical decompensation in patients with cirrhosis using the HepQuant SHUNT test. Aliment Pharmacol Ther. 2021; 53(8):928-38. doi: 10.1111/apt.16283. PubMed PMID: 33556192.
19. Everson G T, Shiffman M L, Hoefs J C, Morgan T R, Sterling R K, Wagner D A, et al. Quantitative liver function tests improve the prediction of clinical outcomes in chronic hepatitis C: Results from the hepatitis C antiviral long-term treatment against cirrhosis trial. Hepatology. 2012; 55(4):1019-29. doi: 10.1002/hep.24752.
20. Lee W M, Dienstag J L, Lindsay K L, Lok A S, Bonkovsky H L, Shiffman M L, et al. Evolution of the HALT-C Trial: pegylated interferon as maintenance therapy for chronic hepatitis C in previous interferon nonresponders. Controlled Clinical Trials. 2004; 25(5):472-92. doi: 10.1016/j.cct.2004.08.003.
21. Campbell I. Chi-squared and Fisher-Irwin tests of two-by-two tables with small sample recommendations. Stat Med. 2007; 26(19):3661-75. doi: 10.1002/sim.2832. PubMed PMID: 17315184.
22. Richardson J T E. The analysis of 2×2 contingency tables-Yet again. Stat Med. 2011; 30(8):890. doi: 10.1002/sim.4116.
23. R Core Team. R: a language and environment for statistical computing. R Foundation for Statistical Computing, Vienna, Austria [Internet]. 2020. Available from: https://www.R-project.org/.
24. HepQuant. RISK ACE Calculators 2024 [12 Sep. 2024]. Available from: www.hepquant.com/risk-ace/.
25. Chang W, Cheng J, Allaire J, Sievert C, Schloerke B, Xie Y, et al. shiny: Web Application Framework for R. R package version 1.8.1.9001. 2024. doi: https://github.com/rstudio/shiny.
26. Malinchoc M, Kamath P S, Gordon F D, Peine C J, Rank J, ter Borg P C J. A model to predict poor survival in patients undergoing transjugular intrahepatic portosystemic shunts. Hepatology. 2000; 31(4):864-71. doi: 10.1053/he.2000.5852.
27. Kim W R, Biggins S W, Kremers W K, Wiesner R H, Kamath P S, Benson J T, et al. Hyponatremia and mortality among patients on the liver-transplant waiting list. N Engl J Med. 2008; 359(10):1018-26. doi: 10.1056/NEJMoa0801209. PubMed PMID: 18768945; PubMed Central PMCID: PMCPMC4374557.
28. Thrift A P, Kanwal F, El-Serag H B. Prediction Models for Gastrointestinal and Liver Diseases: Too Many Developed, Too Few Validated. Clin Gastroenterol Hepatol. 2016; 14(12):1678-80. doi: 10.1016/j.cgh.2016.08.026.
29. Tapper E B, Parikh N D. Mortality due to cirrhosis and liver cancer in the United States, 1999-2016: observational study. BMJ. 2018; 362:k2817. doi: 10.1136/bmj.k2817.
30. McRae M P, Helmke S M, Burton J R, Jr., Everson G T. Compartmental model describing the physiological basis for the HepQuant SHUNT test. Transl Res. 2023; 252:53-63. doi: 10.1016/j.trsl.2022.08.002.
31. Burton J R, Helmke S, Lauriski S, Kittelson J, Everson G T. The within-individual reproducibility of the disease severity index from the HepQuant SHUNT test of liver function and physiology. Transl Res. 2021; 233:5-15. doi: 10.1016/j.trsl.2020.12.010.
32. Helmke S M, McRae M P, Christians U, Shokati T, Everson G T. A Validated LC-MS/MS Assay for the Quantification of Cholate Isotopes in Human Serum. J Appl Lab Med. 2024; 9(6):1028-1039. doi:10.1093/jalm/jfae094
33. McRae M P, Helmke S M, Burton J R, Jr., Everson G T. Compartmental model describing the physiological basis for the HepQuant SHUNT test. Transl Res. 2023; 252:53-63. doi:10.1016/j.trsl.2022.08.002
34. Hassanein T, Keaveny A P, Mantry P, et al. Liver function and portal-systemic shunting quantified by the oral cholate challenge test and risk for large oesophageal varices. Aliment Pharmacol Ther. 2024; 60(2):246-256. doi:10.1111/apt.18054
35. Shiffman M, Reddy K R, Leise M D, et al. Cholate Shunt, Oral Cholate Challenge and Endoscopic Lesions of Portal Hypertension: The SHUNT-V Study. Aliment Pharmacol Ther. 2025; 61(1):75-87. doi:10.1111/apt.18386
36. Kittelson J, McRae M P, Everson G T. Measuring the risk of clinical adverse events (RISK ACE) by quantifying liver function: A patient-centric model. Eur J Intern Med. 2024; 132:160-163. doi:10.1016/j.ejim.2024.11.029
37. Kokkorakis M, Boutari C, Hill M A, et al. Resmetirom, the first approved drug for the management of metabolic dysfunction-associated steatohepatitis: Trials, opportunities, and challenges. Metab Clin Exp. 2024; 154doi:10.1016/j.metabol.2024.155835
38. Harrison S A, Taub R, Neff G W, et al. Resmetirom for nonalcoholic fatty liver disease: a randomized, double-blind, placebo-controlled phase 3 trial. Nature Medicine. 2023/11/01 2023; 29(11):2919-2928. doi:10.1038/s41591-023-02603-1
39. Harrison Stephen A, Bedossa P, Guy Cynthia D, et al. A Phase 3, Randomized, Controlled Trial of Resmetirom in NASH with Liver Fibrosis. N Engl J Med. 2024/02/07 2024; 390(6):497-509. doi:10.1056/NEJMoa2309000
40. Harrison S A, Bashir M R, Guy C D, et al. Resmetirom (MGL-3196) for the treatment of non-alcoholic steatohepatitis: a multicentre, randomised, double-blind, placebo-controlled, phase 2 trial. Lancet. 2019; 394(10213):2012-2024. doi:10.1016/S0140-6736(19)32517-6
21. Harrison S A, Ratziu V, Anstee Q M, et al. Design of the phase 3 MAESTRO clinical program to evaluate resmetirom for the treatment of nonalcoholic steatohepatitis. Aliment Pharmacol Ther. 2024; 59(1):51-63. doi:10.1111/apt.17734
42. Shiffman M, Reddy K R, Liese M D, et al. Cholate Shunt, Oral Cholate Challenge, and Endoscopic Lesions of Portal Hypertension: The SHUNT-V Study. Aliment Pharmacol Ther.; 2025 January; 61 (1):75-87 doi:10.1111/apt.18386
43. Helmke S, Kittelson J, Imperial J C, McRae M P, Everson G T. The Oral Cholate Challenge Test Quantifies Risk for Liver-Related Clinical Outcomes in Primary Sclerosing Cholangitis. Gastro Hep Advances. 2024; 3(7):944-953. doi:10.1016/j.gastha.2024.07.005
44. McRae, M P, Shokati, T, Christians, U, Helmke, S M, Everson, G T. Assessment of the performance od a dual-sample oral cholate challenge test: The HepQuant DuO Test. Clin Chim Acta. 2025 Jun. 15; 574:120325. doi.10.1016/j.cca.2025.120325. Epub 2025 Apr. 20.

1. A method of determining risk for an adverse clinical outcome in a patient having a chronic liver disease, the method comprising
obtaining a baseline test value from a distinguishable cholate challenge test in the patient;
entering the patient baseline test value to a data base comprising comparative distinguishable cholate challenge test values computed from measurements of labeled cholate concentrations in blood samples obtained over time from a known population of chronic liver disease subjects that had been followed prospectively for clinical events; and
computing the individual risk for clinical outcome in the patient using the baseline test value as a predictor, optionally wherein the risk for clinical outcome is displayed over a period of time of at least 2 years, at least 3 years, or at least 4 years.

2. The method of claim 1, wherein the data base comprising comparative test values was prepared from a Cox proportional hazards regression model, or similar statistical methods including Cox proportional hazards regression models with time-varying covariates, parametric survival models, or Kaplan Meier, to predict risk for adverse clinical events (RISK ACE) using the comparative distinguishable cholate challenge test values from the distinguishable cholate challenge test.

3. The method of claim 1, wherein the distinguishable cholate challenge test is selected from the group consisting of a liver disease severity index (DSI) test, cholate SHUNT test, an oral only distinguishable cholate challenge test (DuO), Hepatic Reserve test, portal hepatic filtration rate (HFR_{P}), and systemic hepatic filtration rate (HFRs), optionally wherein the distinguishable cholate challenge test comprises a DSI test or a cholate SHUNT test.

4. The method of claim 1, wherein the patient is suffering from a compensated advanced chronic liver disease (cACLD).

5. The method of claim 1, wherein the clinical outcome is selected from the group consisting of liver-related death, hepatocellular carcinoma (HCC), Child-Pugh progression, variceal hemorrhage, ascites, and hepatic encephalopathy, and optionally, other manifestations of hepatic decompensation selected from the group consisting of jaundice, coagulopathy, nutritional deficiencies, muscle wasting, sepsis, and spontaneous bacterial peritonitis.

6. The method of claim 1, wherein the comparative test values were collected in at least two different time points from the population of known chronic liver disease subjects.

7. The method of claim 1, further comprising
obtaining a follow-up distinguishable cholate challenge test value in the patient after a first period of time and entering the follow-up test value to the data base comprising the comparative test values;
entering date the baseline test value was obtained and date the follow-up test value was obtained; and
computing the individual risk for clinical outcome in the patient using the baseline test value, and subsequent change from baseline in the follow-up test value as predictors.

8. The method of claim 1, further comprising
obtaining a follow-up distinguishable cholate challenge test value in the patient and entering the follow-up test value to the data base comprising the comparative test values;
entering date the baseline test value was obtained and date the follow-up test value was obtained; and
computing the individual risk for clinical outcome in the patient using the baseline test value, and the follow-up test value as predictors.

9. The method of claim 1, further comprising
obtaining a follow-up distinguishable cholate challenge test value in the patient and entering the follow-up test value to the data base comprising the comparative test values;
entering the dates the baseline and follow-up test values were obtained in the patient; and
computing the individual risk for clinical outcome in the patient using the baseline test value and change between the baseline and follow-up test values divided by the time in months between the baseline test and follow-up test to obtain rate of change between baseline and follow-up test values as predictors.

10. The method of claim 1, wherein the distinguishable cholate compound is an isotope labeled distinguishable cholate compound.

11. The method of claim 9, wherein the distinguishable cholate compound is a stable isotope labeled distinguishable cholate compound.

12. The method of claim 11, wherein the stable isotope labeled cholate compound is selected from the group consisting of d4-cholate, d5-cholate, d2 cholate, and ¹³C-cholate, optionally wherein the d4-cholate is 2,2,4,4-d4 cholate, optionally wherein the d5-cholate is 2,2,3,4,4-cholate, and further optionally wherein the 13C cholate is 24-¹³C-cholate.

13. The method of claim 1, wherein the chronic liver disease is selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

14. The method of claim 1, wherein the patient or subject is a human patient or subject.

15. The method of claim 1, comprising
obtaining blood or serum sample concentration data of an orally administered distinguishable cholate compound collected from the patient at two time points after oral administration;
measuring the area under the curve of the blood or serum concentrations of the orally administered distinguishable cholate compound (AUCoral) in the patient comprising
simulating a full oral clearance curve using a compartmental model of oral cholate clearance, the compartmental model comprising body mass index (BMI), body weight (BW), and optionally hematocrit (Hct) input values in the patient, and
calculating the area comprising trapezoidal numerical integration to obtain the AUCoral; and
calculating one or more distinguishable cholate challenge test results in the patient using the AUCoral, wherein the test results are associated with liver function in the patient.

16. The method of claim 15, wherein the obtaining concentration data of the administered distinguishable cholate compound at the two time points comprises
receiving first and second blood or serum samples that had been collected from the patient at first and second time points following a single oral dose of a first distinguishable cholate compound; and
analyzing the samples to obtain the oral concentration data at the first and second time points, optionally
wherein the blood or serum samples had been collected within about 180 minutes, 120 minutes, 90 minutes, or within about 75 minutes, after the oral administration.

17. The method of claim 16, wherein the first and second blood or serum samples had been collected from the subject between at least about 5 min to about 75 min, 10 min to 70 min, 20 min to 60 min, 25 min to 55 min, 30 min to 50 min, 35 to 45 min, or about 40 min apart.

18. The method of claim 16, wherein the first and second blood or serum samples had been collected from the patient at about 20 min and about 60 min following the oral administration, respectively.

19. The method of claim 15, further comprising
estimating an area under the curve of blood or serum concentrations of an intravenously administered distinguishable cholate compound (AUCiv); and
calculating a DSI value or SHUNT test value in the patent using the AUCoral and estimated AUCiv values.

20. The method of claim 19, wherein the estimating the AUCiv comprises a linear regression model, optionally wherein the linear regression model comprises equation 11A:
$${AUC}_{IV} = {β}_{0} + {β}_{BW} \cdot BW + {β}_{PO , 20} \cdot {C}_{PO , 20} + {β}_{PO , 60} \cdot {C}_{PO , 60} + {β}_{HFR , P} \cdot {HFR}_{P} , \qquad Eqn . 11 A$$
wherein
β₀ is an intercept coefficient, optionally wherein the intercept coefficient is 161.972;
B_{BW} is a body weight coefficient, optionally wherein the body weight coefficient is 0.6459;
β_{PO,20} is an orally administered distinguishable cholate concentration coefficient at a first time point, optionally wherein the β_{PO,20} is 16.9249;
C_{PO,20} is the orally administered distinguishable cholate concentration at the first time point;
β_{PO,60} is an orally administered distinguishable cholate concentration coefficient at a second time point, optionally wherein the β_{PO,60} is 89.2405;
C_{PO,60} is an orally administered distinguishable cholate concentration at the second time point; and
β_{HFR, P} is a portal HFR coefficient, optionally wherein the portal FR coefficient is −0.4755.

21. The method of claim 20, wherein the estimating the AUCiv comprises
exponential fitting the intravenous concentration data to a systemic cholate clearance curve comprising fast, moderate, and slow phases of clearance over at least about 180 min after the iv administration of the intravenous dose.

22. The method of claim 21, wherein the fitting to systemic cholate clearance curve fast phase (Y₀) is calculated according to equation 20:
$${Y}_{0} = {C}_{0} \cdot {e}^{- {k}_{fast} \cdot t} , \qquad Eqn . 20$$
wherein
t=time (0 to 20 min);
C₀ is the initial concentration of intravenously administered distinguishable cholate compound;
C₂₀ is the measured 20-minute concentration of intravenously administered distinguishable cholate compound; and
k_{fast} is the rate of elimination in the fast phase estimated by equation 18:
$${k}_{fast} = - \frac{ln ( {C}_{20} ) - ln ( {C}_{0} )}{{T}_{20}} , \qquad Eqn . 18$$
optionally wherein
T₂₀ is the actual time recorded for the 20-minute sample;
C₀ is estimated according to equation 17;
$${C}_{0} = \frac{{D}_{IV}}{{V}_{d} \cdot BW} , \qquad Eqn . 17$$
wherein
D_{IV} is the intravenous dose of third distinguishable cholate;
BW is subject body weight (kg); and
Vd is the volume of distribution (V_{d}) in L per kg body weight, calculated according to equation 16A:
$$TPV = {V}_{d} = \frac{0.07}{\sqrt{BMI / 22}} \cdot ( 1 - Hct ) , \qquad Eqn . 16 A$$
wherein TPV is total plasma volume, BMI is body mass index, and Hct is hematocrit in the subject.

23. The method of claim 21, wherein the fitting to systemic cholate clearance curve moderate phase (Y₁) is calculated according to equation 21:
$${Y}_{1} = \frac{{C}_{20}}{{e}^{- {k}_{mod} \cdot 20}} \cdot {e}^{- {k}_{mod} \cdot t} , \qquad Eqn . 21$$
t=time (20-60 min);
k_{mod} is the rate of elimination in the moderate phase estimated by equation 19:
$${k}_{mod} = - \frac{ln ( {C}_{60} ) - ln ( {C}_{20} )}{{T}_{60} - {T}_{20}} , \qquad Eqn . 19$$
T₂₀ is the actual time recorded for the 20-minute sample;
T₆₀ is the actual time recorded for the 60-minute sample;
C₂₀ is the concentration at 20 minutes;
C₆₀ is the concentration at 60 minutes.

24. The method of claim 21, wherein the fitting to systemic cholate clearance curve slow phase (Y₂) is calculated according to equation 22:
$${Y}_{2} = \frac{{C}_{60}}{{e}^{- {k}_{slow} \cdot {T}_{60}}} \cdot {e}^{- {k}_{slow} \cdot t} , \qquad Eqn . 22$$
wherein
t=time (60-180 min);
C₆₀ is the 60-minute concentration of intravenously administered distinguishable cholate; and
k_{slow} is the rate of elimination in the slow phase estimated by a mean value from a multiplicity of CLD patients, optionally wherein k_{slow} is 0.018 min⁻¹.

25. The method according to claim 21, wherein the areas under each of the three exponential curve fits are calculated by trapezoidal numerical integration and summed to estimate the AUC_{IV}.

26. A computer program product, comprising a non-transitory and tangible computer-readable storage medium whose contents include a program with instructions being executed on a processor so as to perform a method for determining risk for an adverse clinical outcome in a patient having a chronic liver disease, the method comprising
obtaining a baseline test value from a distinguishable cholate challenge test in the patient;
entering the patient baseline test value to a data base comprising comparative distinguishable cholate challenge test values computed from measurements of labeled cholate concentrations in blood samples obtained over time from a known population of chronic liver disease subjects that had been followed prospectively for clinical events; and
computing the individual risk for clinical outcome in the patient using the baseline test value as a predictor, optionally wherein the risk for clinical outcome is displayed over a period of time of at least 2 years, at least 3 years, or at least 4 years.

27. A method of analyzing a response to a treatment of a chronic liver disease in a patient in need thereof, comprising
determining a baseline cholate Disease Severity Index (DSI) value in the patient having a chronic liver disease prior to the treatment;
administering the treatment to the patient for a period of time; and
determining a follow-up cholate Disease Severity Index (DSI) value in the patient after the period of treatment time,
wherein a treatment responder exhibits >2-point decrease in the follow-up DSI value, a stable subject exhibits a ΔDSI within ±2-points, and a non-responder exhibits >2-point increase compared to the baseline DSI value.

28. The method of claim 27, wherein the period of time is from 2 weeks to 104 weeks, 4 weeks to 78 weeks, 8 weeks to 52 weeks, or 28 weeks to 48 weeks.

29. The method of claim 27, wherein the determining the baseline cholate DSI value in the patient comprises
obtaining blood or serum sample concentration data of an orally administered distinguishable cholate compound collected from the patient at two time points after oral administration;
measuring the area under the curve of the blood or serum concentrations of the orally administered distinguishable cholate compound (AUCoral) in the patient comprising
simulating a full oral clearance curve using a compartmental model of oral cholate clearance, the compartmental model comprising body mass index (BMI), body weight (BW), and optionally hematocrit (Hct) input values in the patient, and
calculating the area comprising trapezoidal numerical integration to obtain the AUCoral;
estimating an area under the curve of blood or serum concentrations of an intravenously administered distinguishable cholate compound (AUCiv); and
calculating the DSI value in the patent using the AUCoral and estimated AUCiv values.

30. The method of claim 29, wherein the obtaining concentration data of the orally administered distinguishable cholate compound at the two time points comprises
receiving first and second blood or serum samples that had been collected from the patient at first and second time points following a single oral dose of a first distinguishable cholate compound; and
analyzing the samples to obtain the oral concentration data at the first and second time points, optionally
wherein the blood or serum samples had been collected within about 180 minutes, 120 minutes, 90 minutes, within about 75 minutes, or within about 60 minutes after the oral administration.

31. The method of claim 30, wherein the first and second blood or serum samples had been collected from the subject between at least about 5 min to about 90 min, 10 min to 75 min, 20 min to 60 min, 25 min to 55 min, 30 min to 50 min, 35 to 45 min, or about 40 min apart.

32. The method of claim 31, wherein the first and second blood or serum samples had been collected from the subject at about 20 min and about 60 min following the oral administration, respectively.
